(19) United States
(12) Patent Application Publication
(10) Pub. No.: US 2024/0233872 A9
(43) Pub. Date: Jul. 11, 2024

(54) COMPONENT MIXTURE MODEL FOR TISSUE IDENTIFICATION IN DNA SAMPLES

(71) Applicant: GRAIL, LLC, Menlo Park, CA (US)

(72) Inventors: Aaron Stern, Burlingame, CA (US); Joerg Bredno, Menlo Park, CA (US); Joseph Marcus, Burlingame, CA (US); Oliver Claude Venn, San Francisco, CA (US); Collin Melton, Menlo Park, CA (US)

(21) Appl. No.: 18/489,492

(22) Filed: Oct. 18, 2023

Related U.S. Application Data
(60) Provisional application No. 63/417,616, filed on Oct. 19, 2022.

(51) Int. Cl. G16B 30/10; G16B 20/20; G16H 50/20

(52) U.S. Cl. CPC G16B 30/10; G16B 20/20; G16H 50/20

(57) ABSTRACT

Methods and systems are disclosed for component deconvolution by a mixture model based on methylation information. A mixture model may be trained agnostic of labels or known component contributions. A system generates a methylation signature for each of a plurality of training samples. The methylation signature may be based on a count or a percentage of a methylation variant(s) expressed in the methylation sequence reads of a training sample at each genomic region of a plurality of genomic regions. The system may train the mixture model using maximum likelihood estimation to deconvolve the component contributions. The mixture model may comprise component submodels and a deconvolution submodel. The component submodels predict a component likelihood based on the methylation signature. The deconvolution submodel predicts the component contributions based on the component likelihoods.

### CROSS REFERENCE TO RELATED APPLICATIONS

This application claims the benefit of U.S. Provisional Application No. 63/417,616, filed Oct. 19, 2022, which is incorporated by reference in its entirety.

### BACKGROUND

### Field of Art

Deoxyribonucleic acid (DNA) methylation plays an important role in regulating gene expression. Aberrant DNA methylation has been implicated in many disease processes, including cancer. DNA methylation profiling using methylation sequencing (e.g., whole genome bisulfite sequencing (WGBS)) is increasingly recognized as a valuable diagnostic tool for detection, diagnosis, and/or monitoring of cancer. For example, specific patterns of differentially methylated regions and/or allele specific methylation patterns may be useful as molecular markers for non-invasive diagnostics using circulating cell-free (cf) DNA. When training a cancer classifier, the classifier is limited by the noisiness of the samples. For example, samples may be misdiagnosed, may include confounding tissue signals, or may include multiple genetic signatures from differing clonal populations in a heterogeneous tumor.

The present disclosure is directed to addressing the above-referenced challenge. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

Early detection of a disease state (such as cancer) in subjects is important as it allows for earlier treatment and therefore a greater chance for survival. Sequencing of DNA fragments in cell-free (cf) DNA sample can be used to identify features that can be used for disease classification. For example, in cancer assessment, cell-free DNA based features (such as presence or absence of a somatic variant, methylation status, or other genetic aberrations) from a blood sample can provide insight into whether a subject may have cancer, and further insight on what type of cancer the subject may have. Towards that end, this description includes systems and methods for analyzing cell-free DNA (cfDNA) sequencing data for determining a subject's likelihood of having a disease.

The present disclosure addresses the problems identified above by providing improved systems and methods for cancer classification. The system trains a mixture model to deconvolve component contributions (also referred to as “proportions”) of DNA fragments to the training samples. A mixture model may be trained agnostic of labels or known component proportions. A system generates a methylation signature for each of a plurality of training samples. The methylation signature is based on a count or a percentage of an alternate methylation variant expressed in the methylation sequence reads of a training sample at each genomic region of a plurality of genomic regions. The system may train the mixture model using maximum likelihood estimation to deconvolve the component contributions. The mixture model may comprise component submodels and a deconvolution submodel. The component submodels predict a component likelihood based on the methylation signature. A component likelihood is a predicted likelihood that the nucleic acid molecules represented by the methylation signature originate from a component. When training the mixture model, the number of component submodels may be tuned. The deconvolution submodel predicts the component proportions based on the component likelihoods.

The mixture model may have a variety of applications within the grand scheme of cancer classification.

In one or more embodiments, the mixture model may be utilized for tissue purity assessment. Tissue purity assessment may involve checking whether a sample labeled to have known component contributions indeed has such component contributions. Tissue purity assessment may also include assessing whether a cancer sample has significant tissue contribution for use in training of the cancer classifier. Tissue purity assessment can also be folded into contamination detection. For example, if a sample is said to be derived from a particular tissue, but the predicted tissue proportion is different, then the sample may be deemed contaminated.

In one or more embodiments, the mixture model may be utilized to predict a cancer type of a test subject, e.g., an organ type that is primarily affected. The mixture model may be used to output a predominant component proportion that corresponds to a predicted cancer type. In one or more embodiments, the mixture model may be utilized to quantify cancer signal in a test subject.

In one or more embodiments, the analytics system may utilize the component submodels to determine origination of a methylation sequence read. In one or more embodiments, the analytics system may extract learned information from the mixture model. In one embodiment, the analytics system may learn correlations between methylation variants at genomic regions contributing to component proportions. For example, the analytics system may identify methylation variants that are informative of particular components. In another embodiment, the analytics system may identify methylation variants at genomic regions correlated with non-cancer impurity.

Clause 1. A method for training a machine-learned mixture model for identifying tissue types comprising: obtaining a set of training samples comprising at least one thousand methylation sequence reads derived from sequencing deoxyribonucleic acid (DNA) fragments; modifying each training sample to produce a corresponding sample methylation signature by determining, for each genomic region of a plurality of genomic regions, a first set of methylation sequence reads that overlap the genomic region and a second set of methylation sequence reads that include a methylation variant at the genomic region, the sample methylation signature generated based at least in part on the first sets of methylation sequence reads and the second sets of methylation sequence reads; generating a training set of data comprising the sample methylation signatures; and training the machine-learned mixture model using the training set of data, the machine-learned mixture model configured to identify a contribution of each of a plurality of originating tissue types for DNA fragments in a sample.

Clause 2. The method of clause 1, wherein at least one training sample is known to comprise a first originating tissue type of the plurality of originating tissue types, wherein training the mixture model comprises training the mixture model to identify contribution of the first originating tissue type for DNA fragments in the one training sample.

Clause 3. The method of any of clauses 1-2, wherein at least one training sample is known to have contribution of DNA fragments from each of the plurality of originating tissue types, wherein training the mixture model comprises training the mixture model to identify contribution of each of the plurality of originating tissue types for DNA fragments in the one training sample.

Clause 4. The method of any of clauses 1-3, wherein at least one training sample is one of: a liquid biopsy sample, a tissue biopsy sample, and a purified sample.

Clause 5. The method of any of clauses 1-4, wherein at least one genomic region consists of one CpG site.

Clause 6. The method of any of clauses 1-5, wherein at least one genomic region comprises a plurality of CpG sites.

Clause 7. The method of any of clauses 1-6, further comprising: determining an average sequencing depth for each of an initial set of genomic regions based on the methylation sequence reads of the training samples; and filtering out genomic regions with average sequencing depth below a threshold depth to select the plurality of genomic regions.

Clause 8. The method of any of clauses 1-7, wherein the methylation variant at a genomic region is one of two methylation patterns at the genomic region.

Clause 9. The method of clause 8, wherein the two methylation patterns at one genomic region comprise methylation and unmethylation.

Clause 10. The method of any of clauses 1-9, wherein the methylation variant at a genomic region is one of more than two methylation patterns at the genomic region.

Clause 11. The method of any of clauses 1-10, wherein modifying each training sample to produce the corresponding sample methylation signature further comprises: determining, for each genomic region of the plurality of genomic regions, a third set of methylation sequence reads having a reference state at the genomic region, wherein the reference state is any methylation pattern not belonging to the methylation variant, wherein the sample methylation signature is generated further based on the third set of methylation sequence reads.

Clause 12. The method of any of clauses 1-11, wherein the tissue types include a combination of: a non-cancer impurity; squamous cell cancer tissue; skin carcinoma tissue; melanoma tissue; lung cancer tissue; adenocarcinoma of the lung tissue; squamous carcinoma of the lung tissue; cancer of the peritoneum tissue; gastrointestinal cancer tissue; pancreatic cancer tissue; cervical cancer tissue; ovarian cancer tissue; liver cancer tissue; hepatoma tissue; hepatic carcinoma tissue; bladder cancer tissue; testicular cancer tissue; breast cancer tissue; brain cancer tissue; colon cancer tissue; rectal cancer tissue; colorectal cancer tissue; endometrial or uterine carcinoma tissue; salivary gland carcinoma tissue; kidney or renal cancer tissue; prostate cancer tissue; vulvar cancer tissue; thyroid cancer tissue; anal carcinoma tissue; penile carcinoma tissue; head and neck cancer tissue; esophageal carcinoma tissue; and nasopharyngeal carcinoma (NPC) tissue.

Clause 13. The method of clause 12, wherein the non-cancer impurity comprises one or more of lymphocytes, macrophages, fibroblasts, vascular endothelial cells, or non-cancer tissue.

Clause 14. The method of any of clauses 12-13, wherein the methylation signature for the non-cancer impurity is retrieved from a reference database comprising a plurality of methylation signatures for non-cancer impurity.

Clause 15. The method of any of clauses 1-14, wherein training the machine-learned mixture model is according to a maximum likelihood estimation.

Clause 16. The method of any of clauses 1-15, wherein training the machine-learned mixture model comprises tuning a number of tissue types as one hyperparameter of the machine-learned mixture model.

Clause 17. The method of clause 16, wherein tuning the number of tissue types as one hyperparameter of the machine-learned mixture model comprises: for each number of originating tissue types in the range: training the machine-learned mixture model having the number as the hyperparameter, determining a maximum likelihood by cross-validating the trained machine-learned mixture model with a holdout set of samples, and implementing a penalization to the maximum likelihood based on the number; and selecting an optimal number from the range as the hyperparameter based on penalized maximum likelihoods.

Clause 18. The method of any of clauses 1-17, wherein training the machine-learned mixture model comprises training according to one or more machine-learning algorithms.

Clause 19. The method of any of clauses 1-18, wherein the machine-learned mixture model comprises a first set of tissue type models, each tissue type model modeling methylation signature of DNA fragments of an originating tissue type, and wherein training the machine-learned mixture model comprises training the first set of tissue type models.

Clause 20. The method of clause 19, wherein training the first submodels comprises training each tissue type model according to a Beta distribution.

Clause 21. The method of any of clauses 1-20, wherein the machine-learned mixture model comprises a deconvolution model for deconvolving the contributions of the originating tissue types for each training sample, wherein training the machine-learned mixture model comprises training the deconvolution model.

Clause 22. The method of clause 21, wherein training the deconvolution model comprises training the deconvolution model according to a binomial distribution.

Clause 23. The method of any of clauses 1-22, wherein the machine-learned mixture model comprises a first tier of one or more submodels to predict contributions of macro originating tissue types and a second tier of one or more submodels to predict contributions of originating tissue types under the macro tissue types, and wherein one first tier submodel predicts a contribution of one macro tissue type and a set of one or more second tier submodels predicts contributions of a set of tissue types under the one macro tissue type equaling the contribution of the one macro tissue type.

Clause 24. A method for identifying contributions of originating tissue types for DNA fragments in a test sample comprising: obtaining the test sample comprising at least one thousand methylation sequence reads for DNA fragments in the test sample; for each of a plurality of genomic regions: determining a first set of methylation sequence reads overlapping the genomic region, and determining a second set of methylation sequence reads having an alternative methylation signature at the genomic region, generating a sample methylation signature for the training sample based on the first sets of methylation sequence reads and the second sets of methylation sequence reads across the plurality of genomic regions; and applying a machine-learning mixture model to the sample methylation signature to identify a contribution of each originating tissue type for DNA fragments in the test sample, optionally wherein the machine-learning mixture model is trained according to the method of any of clauses 1-23.

Clause 25. The method of clause 24, further comprising: reporting the identified contributions of the originating tissue types to a client device.

Clause 26. The method of any of clauses 24-25, further comprising: identifying the originating tissue type with the largest contribution, and reporting a treatment recommendation for treating cancer with primary origin of the identified originating tissue type.

Clause 27. The method of any of clauses 24-26, further comprising: comparing the contributions of originating tissue types to prior contributions of originating tissue types predicted before a treatment; determining treatment efficacy based on the comparison; and reporting a treatment recommendation based on the treatment efficacy.

Clause 28. The method of any of clauses 24-27, further comprising: determining success of the treatment if the contribution of a first originating tissue type is smaller than a prior contribution of the first originating tissue type predicted before the treatment; and reporting the success of the treatment.

Clause 29. The method of any of clauses 24-28, further comprising: determining failure of the treatment if the contribution of a first originating tissue type is larger than a prior contribution of the first originating tissue type predicted before the treatment; and reporting the failure of the treatment.

Clause 30. The method of clause 29, further comprising: reporting a treatment modification based on the failure of the treatment.

Clause 31. The method of any of clauses 24-30, wherein the test sample is predicted to have cancer.

Clause 32. The method of clause 31, wherein the test sample is predicted to have cancer by a cancer classifier.

Clause 33. The method of clause 32, wherein the cancer classifier is trained on a cancer cohort of training samples and a non-cancer cohort of training samples, wherein each training sample from the cancer cohort and the non-cancer cohort comprises at least one thousand methylation sequence reads for DNA fragments in the training sample.

Clause 34. The method of clause 24, further comprising: applying the machine-learning mixture model to the sample methylation signature to determine a subset of methylation sequence reads originating from a non-cancer impurity type; excluding the subset of methylation sequence reads originating from the non-cancer impurity type resulting in a feature set of methylation sequence reads; and applying a cancer classifier to the feature set of methylation sequence reads to predict cancer in the test sample.

Clause 35. A method for training a cancer classifier comprising: obtaining a cancer cohort of training samples and a non-cancer cohort of training samples, wherein each training sample from the cancer cohort and the non-cancer cohort comprises at least one thousand methylation sequence reads for DNA fragments in the training sample; generating a sample methylation for each training sample by: for each of a plurality of genomic regions: determining a first set of methylation sequence reads overlapping the genomic region, determining a second set of methylation sequence reads having an alternative methylation signature at the genomic region, and wherein the sample methylation signature is based in part on the first sets of methylation sequence reads and the second sets of methylation sequence reads, and applying a machine-learning mixture model, optionally trained according to the method of any of clauses 1-23, to the sample methylation signature of each training sample in the cancer cohort to identify a subset of methylation sequence reads originating from a non-cancer impurity type; excluding, for each training sample in the cancer cohort, the subset of methylation sequence reads originating from the non-cancer impurity type resulting in a feature set of methylation sequence reads; generating, for each training sample in the non-cancer cohort, a feature set of methylation sequence reads; and training the cancer classifier with the feature sets of methylation sequence reads for the training samples from the cancer cohort and the feature sets of methylation sequence reads for the training samples from the non-cancer cohort.

Clause 36. The method of clause 35, wherein the cancer classifier is trained as a machine-learning model.

Clause 37. A method for training a cancer classifier, the method comprising: obtaining, for each training sample of a plurality of training samples, a set of methylation sequence reads derived from sequencing DNA fragments of the training sample, wherein the plurality of training samples includes training samples obtained from a first cohort of subjects diagnosed with cancer and training samples obtained from a second cohort of subjects not diagnosed with cancer; for each training sample in the first cohort and for each methylation sequence read of the training sample: applying a tissue type model trained to predict a likelihood that the methylation sequence read is derived from non-cancer tissue, and excluding the methylation sequence read if the predicted likelihood is above a threshold; generating, for each training sample in the first cohort, a methylation signature based on non-excluded methylation sequence reads of the training sample; generating, for each training sample in the second cohort, a methylation signature based on methylation sequence reads of the training sample; and training a cancer classifier to detect a presence of cancer signal with the methylation signatures of the training samples.

Clause 38. The method of clause 37, wherein the tissue type model is trained according to a Beta distribution using methylation signatures of non-cancer training samples generated from methylation sequence reads derived from sequencing of DNA fragments of the non-cancer training samples.

Clause 39. The method of clause 38, wherein the methylation signatures of the non-cancer training samples are retrieved from a reference database.

Clause 40. The method of any of clauses 37-39, wherein the cancer classifier is trained as a machine-learning model.

Clause 41. The method of any of clauses 37-40, wherein the methylation signatures are based on identification of one or more methylation variants at each genomic region of a plurality of genomic regions.

Clause 42. A method for identifying an originating tissue type for a DNA fragment comprising: obtaining a methylation sequence read for the DNA fragment, the methylation sequence read comprising a methylation signature over one or more genomic regions; predicting a likelihood that the methylation signature of the methylation sequence read is from each of a plurality of originating tissue types by applying each of a plurality of tissue type models to the methylation signature, wherein the plurality of tissue type models is part of a mixture model, optionally trained according to the method of any of clauses 1-23; determining that the DNA fragment is derived from the originating tissue type with the largest likelihood; and returning a report of the determination.

Clause 43. A non-transitory computer-readable storage medium storing instructions that, when executed by a computer processor, cause the computer processor to perform the method of any of clauses 1-42.

Clause 44. A system comprising: a computer processor; and the non-transitory computer-readable storage medium of clause 43.

Clause 45. A computer-program product comprising a non-transitory computer-readable storage medium storing a machine-learning mixture model for predicting a proportion of tissue types from a test sample, wherein the product is made by the method of any of clauses 1-23.

Clause 46. A computer-program product comprising a non-transitory computer-readable storage medium storing a machine-learning cancer classifier for predicting cancer in a test sample, wherein the product is made by the method of any of clauses 33-41.

Clause 47. A treatment kit comprising: a collection vessel for collecting a DNA sample from a subject; optionally, one or more reagents for isolating DNA fragments in the DNA sample; optionally, one or more probes targeting one or more genomic loci determined to be indicative of cancer status; and the non-transitory computer-readable storage medium of clause 43 or the computer program product of claim 45 or 46.

### BRIEF DESCRIPTION OF DRAWINGS

FIG. 1 is an exemplary flowchart describing an overall workflow of cancer classification of a sample, according to one or more embodiments.

FIG. 2A is an exemplary flowchart of devices for sequencing nucleic acid samples according to one or more embodiments.

FIG. 2B is a block diagram of an analytics system for processing DNA samples according to one or more embodiments.

FIG. 3A is an exemplary flowchart describing a process of sequencing a fragment of cell-free (cf) DNA to obtain a methylation state vector, according to one or more embodiments.

FIG. 3B is an exemplary illustration of the process of FIG. 2A of sequencing a fragment of cell-free (cf) DNA to obtain a methylation state vector, according to one or more embodiments.

FIG. 4A is an exemplary flowchart describing a process of training a mixture model to predict component proportions in a sample, according to one or more embodiments.

FIG. 4B is an exemplary flowchart describing a process of deploying a mixture model to predict component proportions in a sample, according to one or more embodiments.

FIG. 5A illustrates methylation features that can be derived from a single CpG site as a genomic region, according to one or more embodiments.

FIG. 5B illustrates methylation features that can be derived from multiple CpG sites as a genomic region, according to one or more embodiments.

FIG. 6 illustrates an example methylation signature matrix used in training the mixture model, according to an example implementation.

FIG. 7 illustrates an exemplary architecture of the mixture model, according to one or more embodiments.

FIG. 8A is an exemplary flowchart describing a process of generating a control group data structure for determining anomalously methylated fragments, according to one or more embodiments.

FIG. 8B is an exemplary flowchart describing a process of determining a fragment to be anomalously methylated based on the control group data structure, according to one or more embodiments.

FIG. 9A is an exemplary flowchart describing a process of training a cancer classifier, according to one or more embodiments.

FIG. 9B illustrates an example generation of feature vectors used for training the cancer classifier, according to one or more embodiments.

FIG. 10 is an example result of a two-component mixture model, according to an example implementation.

FIG. 11 is an example result of a five-component mixture model, according to an example implementation.

FIG. 12 is an example result of a mixture model deconvolving breast tissue positive hormone receptor status, breast tissue negative hormone receptor status, prostate tissue, and uterine tissue, according to an example implementation.

FIG. 13A is an example result of a mixture model deconvolving non-cancer impurity from colorectal tissue in colorectal samples, according to an example implementation.

FIG. 13B is an example result of a mixture model deconvolving non-cancer impurity from bladder tissue in bladder samples, according to an example implementation.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

Early detection and classification of cancer is an important technology. Being able to detect cancer before it becomes symptomatic is beneficial to all parties involved, including patients, doctors, and loved ones. For patients, early cancer detection allows them a greater chance of a beneficial outcome; for doctors, early cancer detection allows more pathways of treatment that may lead to a beneficial outcome; for loved ones, early cancer detection increases the likelihood of not losing friends and family to the disease.

Recently, early cancer detection technology has progressed towards analyzing genetic fragments (e.g., DNA) in a person's, for example, blood to determine if any of those genetic fragments originate from cancer cells. These new techniques allow doctors to identify a cancer presence in a patient that may not be detectable otherwise, e.g., in conventional screening processes. For instance, consider the example of a person at high risk for breast cancer. Traditionally, this person will regularly visit their doctor for a mammogram, which creates an image of their breast tissue (e.g., taking x-ray images) that a doctor uses to identify cancerous tissue. Unfortunately, with even the highest resolution mammograms, doctors are only able to identify tumors once they are approximately a millimeter in size. This means that the cancer has been present for some time in the person and has gone undiagnosed and untreated. Visual determinations like this are typical for most cancers—that is, only identifiable once it has grown to a sufficient size to be detected by some sort of imaging technology.

Cancer detection using analysis of genetic fragments in a patient's, e.g., blood alleviates this issue. To illustrate, cancer cells will start sloughing DNA fragments into a person's bloodstream as soon as they form. This occurs when there are very few of the cancer cells, and before they would be visible with imaging techniques. With the appropriate methods, therefore, a system that analyzes DNA fragments in the bloodstream could identify cancer presence in a person based on sloughed cancer DNA fragments, and, more importantly, the system could do so before the cancer is identifiable using more traditional cancer detection techniques.

Cancer detection based on the analysis of DNA fragments is enabled by next-generation sequencing (“NGS”) techniques. NGS, broadly, is a group of technologies that allows for high throughput sequencing of genetic material. As discussed in greater detail herein, NGS largely consists of (1) sample preparation, (2) DNA sequencing, and (3) data analysis. Sample preparation is the laboratory methods necessary to prepare DNA fragments for sequencing, sequencing is the process of reading the ordered nucleotides in the samples, and data analysis is processing and analyzing the genetic information in the sequencing data to identify cancer presence.

While these steps of NGS may help enable early cancer detection, they also introduce their own complex, detrimental problems to cancer detection and, therefore, any improvements to sample preparation, DNA sequencing, and/or data analysis, including the pre-processing, algorithmic processing, and summary or presentation of predications or conclusions, results in an improvement to cancer detection technologies and early cancer detection more generally.

To illustrate, as an example, problems introduced in (1) sample preparation include DNA sample quality, sample contamination, fragmentation bias, and accurate indexing. Remedying these problems would yield better genetic data for cancer detection.

Similarly, problems introduced in (2) sequencing include, for example, errors in accurate transcribing of fragments (e.g., reading an “A” instead of a “C”, etc.), incorrect or difficult fragment assembly and overlap, disparate coverage uniformity, sequencing depth vs. cost vs. specificity, and insufficient sequencing length. Again, remedying any of these problems would yield improved genetic data for cancer detection.

The problems in (3) data analysis are the most daunting and complex. The introduced challenges stem from the vast amounts of data created by NGS sequencing techniques. Sequencing data for a single sample can be on the order of hundreds of thousands (up to millions) of sequence reads, amounting to terabytes of data. Multiply that by the thousands (up to tens of thousands) of samples which are collected for use in training of the analytical models. Effectively and efficiently analyzing that amount of data is both procedurally and computationally demanding. For instance, analyzing NGS sequencing involves several baseline processing steps such as, e.g., aligning reads to one another, aligning and mapping reads to a reference genome, de-duping duplicative reads, detecting contamination of a sample, identifying and calling variant genes, identifying and calling abnormally methylated genes, generating functional annotations, etc. Performing any of these processes on terabytes of genetic data is computationally expensive for even the most powerful of computer architectures, and completely impossible for a normal human mind. Additionally, with the genetic sequencing data derived from the error-prone processes of sample preparation and sequence reading, large portions of the resulting genetic data may be low-quality or unusable for cancer identification. For example, large amounts of the genetic data may include contaminated samples, transcription errors, mismatched regions, overrepresented regions, etc. and may be unsuitable for high accuracy cancer detection. Identifying and accounting for low quality genetic data across the vast amount of genetic data obtained from NGS sequencing is also procedurally and computationally rigorous to accomplish and is also not practically performable by a human mind. Overall, any process created that leads to more efficient processing of large array sequencing data would be an improvement to cancer detection using NGS sequencing. Moreover, such processes were crafted as a solution to the various hurdles created in NGS sequences, and as such are non-routine and unconventional activity in the technical field of endeavor.

Particularly, under (3) data analysis, accurate identification of anomalous DNA from NGS data to identify a cancer presence is also a difficult task-at-hand. To be effective, algorithms are sought to compensate for, e.g., errors generated by sample preparation and sequencing, and to overcome the large-scale data analysis problems accompanying NGS techniques. That is, designing a machine learning model or models, or other computational processing algorithms, that enable early cancer detection based on next generation sequencing techniques must be configured to account for the problems that those techniques create. Some of those techniques and models are discussed hereinbelow and particular improvements to state-of-the-art techniques and models are further discussed. Furthermore, such techniques are non-routine and unconventional activity in the technical field of endeavor.

One particular challenge arises with tissue samples. Generally, the process of collecting tissue samples entails performing a tissue biopsy to remove tissue from the subject. The tissue is thinly sliced to be put on a slide, stained (e.g., with hematoxylin and eosin (H&E)), annotated by a pathologist, and dissected based on the pathologist annotations. The dissected portions are used to isolate nucleic acid fragments pertaining to that tissue, e.g., through lysing of the cells and isolation of the nucleic acid fragments. Such a process relies on the precise annotation of the pathologist as well as the precision in dissection of the target portions to source the nucleic acid fragments. Various points of this process can introduce impurity in the tissue sample. For example, if the annotation and/or the dissection is imprecise, the dissected portion may be overinclusive including tissue beyond the targeted portion. In cancer tissue samples, this may result in the tissue sample including tumorous tissue and healthy tissue, thereby muddying the purity of the tissue sample. Or in other cases, the tissue sample may include additional tissue types that were not targeted, also muddying the purity of the sample.

The invention(s) describe a mixture model that can assess tissue purity in a sample, thus overcoming the challenge described above. Samples deemed impure, i.e., having higher than an acceptable tolerance of contamination, can be removed from the training data sets to prevent skewing the downstream analyses. For example, when training a cancer classifier to classify a tumor sample between differing cancer types, the mixture model can assess purity of cancer samples used in training of the cancer classifier.

Another particular challenge arises in the context of intra-tumor heterogeneity. In such, a cancer sample that originates from a subject diagnosed with cancer may include a heterogeneous tumor. Such a tumor is a mix of two or more cancer subtypes, each potentially having its own tumor biology and/or genetic mutations. Training with such samples under the guise of a single tumor biology can blur classification lines, skewing the predictive accuracy of the analytical model(s).

The invention(s) describe a mixture model that can parse different cancer subtypes having differing methylation signatures in a heterogeneous tumor sample. The mixture model may be trained to deconvolve the varying subtypes in the heterogeneous tumor sample. Samples having substantial portions of two or more cancer subtypes may be withheld from use in training of the analytical models. In other example applications, the mixture model may be trained to predict cancer subtypes, e.g., as a downstream analysis upon detecting likelihood of cancer in a test sample.

### I.A. Cancer Classification Workflow

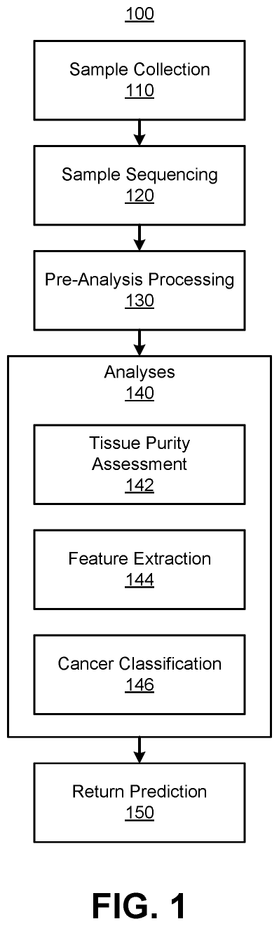

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, ethnicity, smoking status, any prior diagnoses, etc.

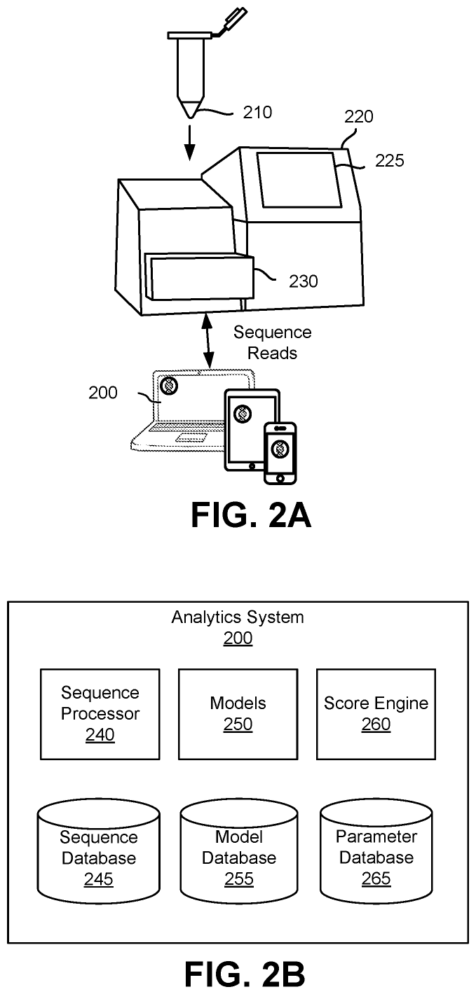

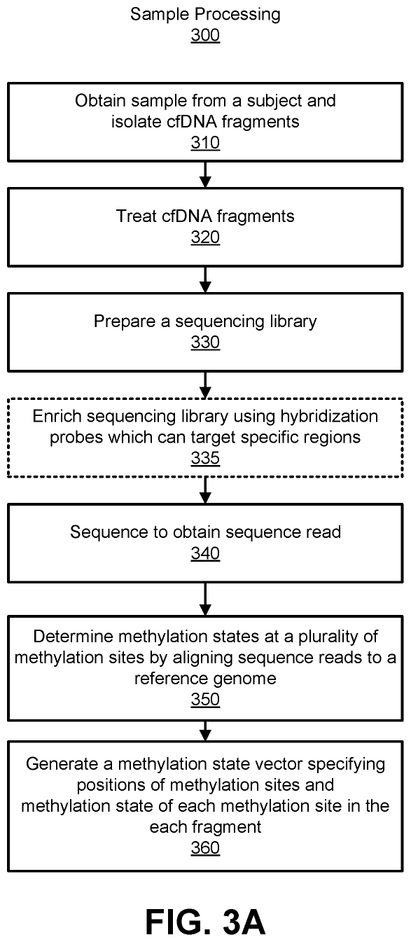

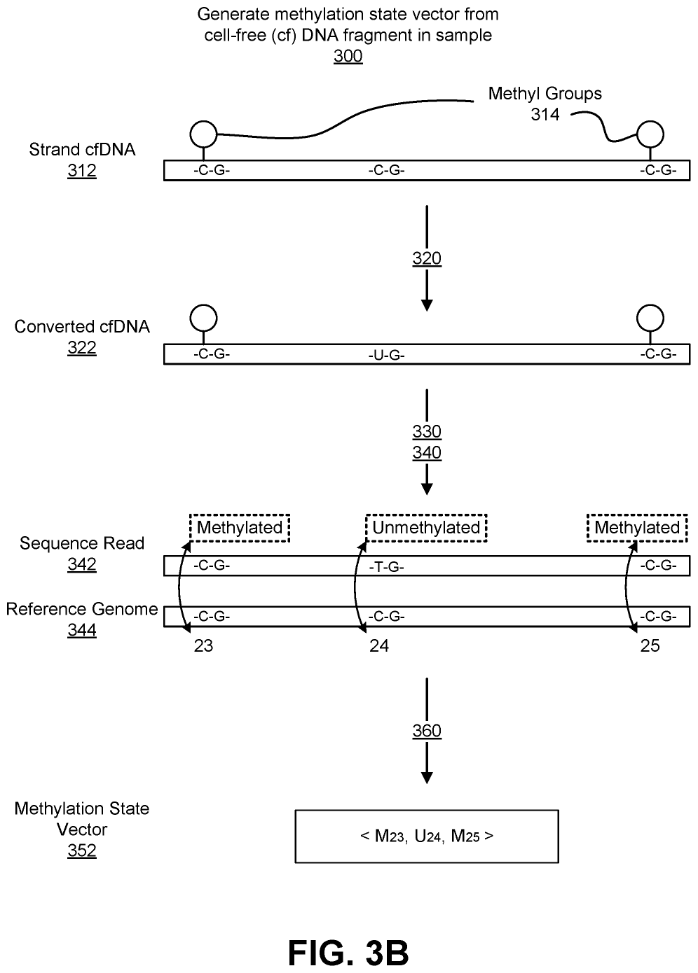

A sequencing device performs sample sequencing 120. A lab clinician may perform one or more processing steps to the sample in preparation of sequencing. Once prepared, the clinician loads the sample in the sequencing device. An example of devices utilized in sequencing is further described in conjunction with FIGS. 2A & 2B. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and next-generation sequencing. Sequencing may be whole-genome sequencing or targeted sequencing with a target panel. In the context of DNA methylation, bisulfite sequencing (e.g., further described in FIGS. 3A & 3B) can determine methylation status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

An analytics system performs pre-analysis processing 130. An example analytics system is described in FIG. 2B. Pre-analysis processing 130 may include, but not limited to, de-duplication of sequence reads, determining metrics relating to coverage, determining whether the sample is contaminated, removal of contaminated fragments, calling sequencing error, etc.

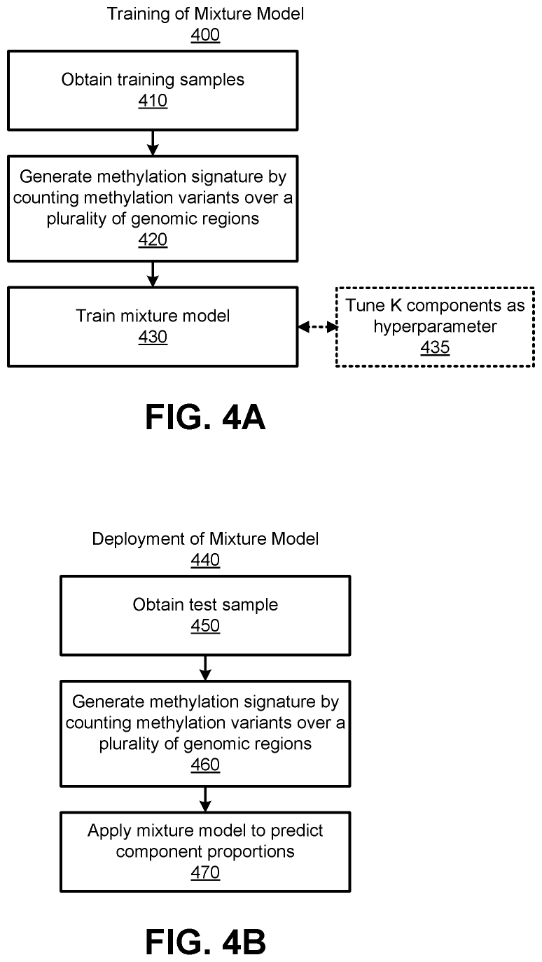

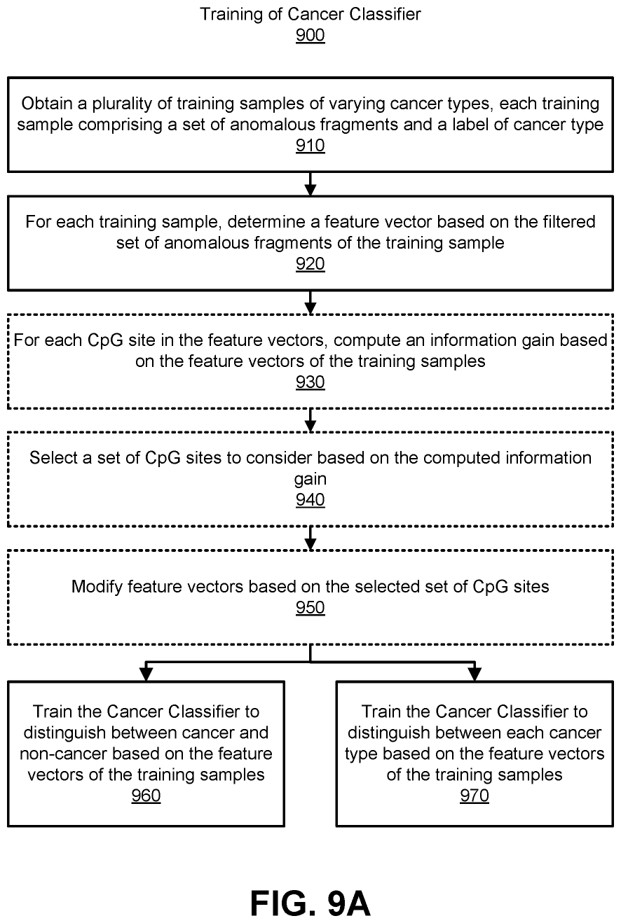

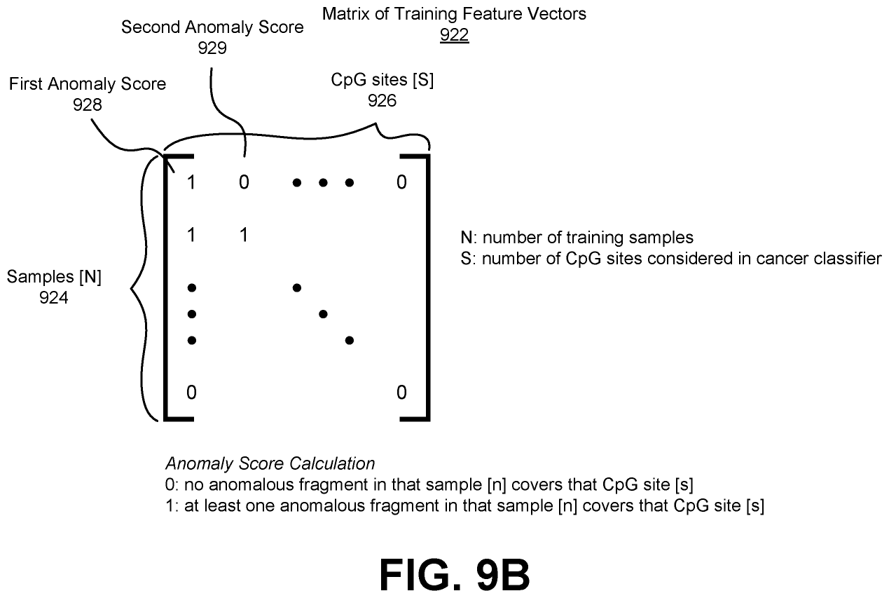

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide polymorphisms (SNPs), insertions or deletions (indels), other types of genetic mutation, etc. In the context of methylation, analyses 140 may include tissue purity assessment 142 (e.g., further described in FIGS. 4A, 4B, 5A, 5B, 6, and 7), feature extraction 144, and applying a cancer classifier 146 to determine a cancer prediction (e.g., further described in FIGS. 9A & 9B). Tissue purity assessment 142 involves applying a mixture model to deconvolute proportions of tissue components contributing DNA fragments to the sample. In general, tissue purity assessment 142 may be used to determine what proportion of methylation sequence reads for a sample were shed from cancerous tissue compared to a proportion of methylation sequence reads from non-cancer cells. Tissue purity assessment 142 may be particularly useful in deconvolving heterogeneous tumors that comprise multiple clonal populations with potentially distinct genetic signatures. The mixture model may also be applied to quantify cancer signal or grade cancer status based on the determined proportions. The cancer classifier 146 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for, cancer stage, etc. The value may indicate a likelihood of a particular cancer state, e.g., a likelihood of cancer, and/or a likelihood of a particular cancer type.

The analytics system returns the prediction 150 to the healthcare provider. The prediction 150 may include binary prediction of presence or absence of cancer, a particular cancer type, cancer stage, tissue proportions, etc. The healthcare provider may establish or adjust a treatment plan based on the returned prediction 150. Optimization of treatment is further described in Section V.C. Treatment.

### I.B. Methylation Overview

According to the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy individuals, may provide insight into a subject's cancer status. As is well known in the art, DNA methylation anomalies (compared to healthy controls) can cause different effects, which may contribute to cancer. Various challenges arise in the identification of anomalously methylated cfDNA fragments. First off, determining a DNA fragment to be anomalously methylated can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### I.C. Definitions

The term “cell free nucleic acid” or “cfNA” refers to nucleic acid fragments that circulate in an individual's body (e.g., blood) and originate from one or more healthy cells and/or from one or more unhealthy cells (e.g., cancer cells). The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “anomalous fragment,” “anomalously methylated fragment,” or “fragment with an anomalous methylation pattern” refers to a fragment that has anomalous methylation of CpG sites. Anomalous methylation of a fragment may be determined using probabilistic models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “anomaly score” refers to a score for a CpG site based on a number of anomalous fragments (or, in some embodiments, UFXMs) from a sample overlaps that CpG site. The anomaly score is used in context of featurization of a sample for classification.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which can reflect a biological state associated with the subject, and that includes cell-free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “cancer” or “tumor” refers to an abnormal mass of tissue in which the growth of the mass surpasses and is not coordinated with the growth of normal tissue.

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Anomalous cfDNA methylation can be identified as hypermethylation or hypomethylation, both of which may be indicative of cancer status. DNA methylation anomalies (compared to healthy controls) can cause different effects, which may contribute to cancer. The principles described herein are equally applicable for the detection of methylation in a CpG context and non-CpG context, including non-cytosine methylation. Further, the methylation state vectors may contain elements that are generally vectors of sites where methylation has or has not occurred (even if those sites are not CpG sites specifically).

As used interchangeably herein, the term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

As used herein, the term “methylation variant” refers to a distinct methylation pattern in a set of k (e.g., 2, 3, 4, 5, 6, 7, 8, 9, 10, 15, 25, etc.) contiguous CpG sites. The methylation variants may be used to distinguish DNA derived from a specific cancer type from non-cancer plasma. A given methylation variant has a single defined “variant state” which is a single specific and detectable pattern of methylation states. A “reference state” is any pattern of methylation states that is not a variant state. As one or more examples, a set of 5 contiguous CpGs can have a variant state of 5 methylated CpGs (e.g., MMMMM, where “M” denotes methylated), another variant state of 5 unmethylated CpGs (e.g., UUUUU, where “U” denotes unmethylated), or a third variant state including a mixture of methylation states (e.g., UMMMU). The reference states can include all other patterns not deemed to be one of the variant states.

As used herein, the term “true positive” (TP) refers to a subject having a condition. “True positive” can refer to a subject that has a tumor, a cancer, a pre-cancerous condition (e.g., a pre-cancerous lesion), a localized or a metastasized cancer, or a non-malignant disease. “True positive” can refer to a subject having a condition and is identified as having the condition by an assay or method of the present disclosure. As used herein, the term “true negative” (TN) refers to a subject that does not have a condition or does not have a detectable condition. True negative can refer to a subject that does not have a disease or a detectable disease, such as a tumor, a cancer, a pre-cancerous condition (e.g., a pre-cancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 450 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequencing depth,” is interchangeably used with the term “coverage” and refers to the number of times a locus is covered by a consensus sequence read corresponding to a unique nucleic acid target molecule aligned to the locus; e.g., the sequencing depth is equal to the number of unique nucleic acid target molecules covering the locus. The locus can be as small as a nucleotide, or as large as a chromosome arm, or as large as an entire genome. Sequencing depth can be expressed as “Y×”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a locus or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus, or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a woman or a child). A subject from whom a sample is taken, or is treated by any of the methods or compositions described herein can be of any age and can be an adult, infant or child.

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” or “originating tissue type” can be used to refer to a tissue from which a cell-free nucleic acid molecule or fragment originates. In one example, viral nucleic acid fragments can be derived from blood tissue. In another example, viral nucleic acid fragments can be derived from tumor tissue.

As used herein, the term “genomic” refers to a characteristic of the genome of an organism. Examples of genomic characteristics include, but are not limited to, those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### I.D. Example Analytics System

FIG. 2A is an exemplary flowchart of devices for sequencing nucleic acid samples according to one or more embodiments. This illustrative flowchart includes devices such as a sequencer 220 and an analytics system 200. The sequencer 220 and the analytics system 200 may work in tandem to perform one or more steps in the processes.

In various embodiments, the sequencer 220 receives an enriched nucleic acid sample 210. As shown in FIG. 2A, the sequencer 220 can include a graphical user interface 225 that enables user interactions with particular tasks (e.g., initiate sequencing or terminate sequencing) as well as one more loading stations 230 for loading a sequencing cartridge including the enriched fragment samples and/or for loading necessary buffers for performing the sequencing assays. Therefore, once a user of the sequencer 220 has provided the necessary reagents and sequencing cartridge to the loading station 230 of the sequencer 220, the user can initiate sequencing by interacting with the graphical user interface 225 of the sequencer 220. Once initiated, the sequencer 220 performs the sequencing and outputs the sequence reads of the enriched fragments from the nucleic acid sample 210.

In some embodiments, the sequencer 220 is communicatively coupled with the analytics system 200. The analytics system 200 includes some number of computing devices used for processing the sequence reads for various applications such as assessing methylation status at one or more CpG sites, variant calling or quality control. The sequencer 220 may provide the sequence reads in a BAM file format to the analytics system 200. The analytics system 200 can be communicatively coupled to the sequencer 220 through a wireless, wired, or a combination of wireless and wired communication technologies. Generally, the analytics system 200 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide base and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 200 may label a sequence read with one or more genes that align to the sequence read. In one embodiment, fragment length (or size) can be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome can represent the likely location within the reference genome to which the nucleic acid fragment corresponds. An output file having SAM (sequence alignment map) format or BAM (binary) format may be generated and output for further analysis.

FIG. 2B is a block diagram of an analytics system 200 for processing DNA samples according to one or more embodiments. The analytics system implements one or more computing devices for use in analyzing DNA samples. The analytics system 200 includes a sequence processor 240, sequence database 245, model database 255, models 250, parameter database 265, and score engine 260. In some embodiments, the analytics system 200 performs some or all of the processes described throughout this disclosure.

The sequence processor 240 generates methylation state vectors for fragments from a sample. At each CpG site on a fragment, the sequence processor 240 generates a methylation state vector for each fragment specifying a location of the fragment in the reference genome, a number of CpG sites in the fragment, and the methylation state of each CpG site in the fragment whether methylated, unmethylated, or indeterminate via the process 200 of FIG. 2A. The sequence processor 240 may store methylation state vectors for fragments in the sequence database 245. Data in the sequence database 245 may be organized such that the methylation state vectors from a sample are associated to one another.

Further, multiple different models 250 may be stored in the model database 255 or retrieved for use with test samples. Generally, a model receives an input and generates an output based on a function that operates on the input according to one or more parameters. In one example, a model is a trained mixture model for deconvolving component proportions based on a sample's methylation signature. The mixture model may comprise various submodels each with its own parameters and function(s). In another example, a model is a trained cancer classifier for determining a cancer prediction for a test sample using a feature vector derived from anomalous fragments. The training and use of the cancer classifier will be further discussed in conjunction with Section IV. Cancer Classifier for Determining Cancer. The analytics system 200 may train the one or more models 250 and store various trained parameters in the parameter database 265. The analytics system 200 stores the models 250 along with functions in the model database 255.

During inference, the score engine 260 uses the one or more models 250 to return outputs. The score engine 260 accesses the models 250 in the model database 255 along with trained parameters from the parameter database 265. According to each model, the score engine receives an appropriate input for the model and calculates an output based on the received input, the parameters, and a function of each model relating the input and the output. In some use cases, the score engine 260 further calculates metrics correlating to a confidence in the calculated outputs from the model. In other use cases, the score engine 260 calculates other intermediary values for use in the model.

### II. Methylation Sequencing of DNA Fragments

FIG. 3A is an exemplary flowchart describing a process 300 of sequencing a fragment of cfDNA to obtain a methylation state vector, according to one or more embodiments. In order to analyze DNA methylation, an analytics system first obtains 310 a sample from an individual comprising a plurality of cfDNA molecules. In additional embodiments, the process 300 may be applied to sequence other types of DNA molecules. The process 300 is an embodiment of sample sequencing 120 of FIG. 1.

From the sample, the analytics system can isolate 310 each cfDNA molecule. The cfDNA molecules can be treated 320 to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted cfDNA molecules, a sequencing library can be prepared 330. During library preparation, unique molecular identifiers (UMI) can be added to the nucleic acid molecules (e.g., DNA molecules) through adapter ligation. The UMIs can be short nucleic acid sequences (e.g., 4-10 base pairs) that are added to ends of DNA fragments (e.g., DNA molecules fragmented by physical shearing, enzymatic digestion, and/or chemical fragmentation) during adapter ligation. UMIs can be degenerate base pairs that serve as a unique tag that can be used to identify sequence reads originating from a specific DNA fragment. During PCR amplification following adapter ligation, the UMIs can be replicated along with the attached DNA fragment. This can provide a way to identify sequence reads that came from the same original fragment in downstream analysis.

Optionally, the sequencing library may be enriched 335 for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. Hybridization probes can be tiled across one or more target sequences at a coverage of 1×, 2×, 3×, 4×, 5×, 6×, 7×, 8×, 9×, 10×, or more than 10×. For example, hybridization probes tiled at a coverage of 2× comprises overlapping probes such that each portion of the target sequence is hybridized to 2 independent probes. Hybridization probes can be tiled across one or more target sequences at a coverage of less than 1×.

In one embodiment, the hybridization probes are designed to enrich for DNA molecules that have been treated (e.g., using bisulfite) for conversion of unmethylated cytosines to uracils. During enrichment, hybridization probes (also referred to herein as “probes”) can be used to target and pull down nucleic acid fragments informative for the presence or absence of cancer (or disease), cancer status, or a cancer classification (e.g., cancer class or tissue of origin). The probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. The probes can be designed based on a methylation site panel. The probes can be designed based on a panel of targeted genes to analyze particular mutations or target regions of the genome (e.g., of the human or another organism) that are suspected to correspond to certain cancers or other types of diseases. Moreover, the probes may cover overlapping portions of a target region.

Once prepared, the sequencing library or a portion thereof can be sequenced 340 to obtain a plurality of sequence reads. The sequence reads may be in a computer-readable, digital format for processing and interpretation by computer software. The sequence reads may be aligned to a reference genome to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. A sequence read can be comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome can represent the likely location within the reference genome to which the nucleic acid fragment corresponds. An output file having SAM (sequence alignment map) format or BAM (binary) format may be generated and output for further analysis such as methylation state determination.

From the sequence reads, the analytics system determines 350 a location and methylation state for each CpG site based on alignment to a reference genome. The analytics system generates 360 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states can be states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses.

FIG. 3B is an exemplary illustration of the process 300 of FIG. 3A of sequencing a cfDNA molecule to obtain a methylation state vector, according to one or more embodiments. As an example, the analytics system receives a cfDNA molecule 312 that, in this example, contains three CpG sites. As shown, the first and third CpG sites of the cfDNA molecule 312 are methylated 314. During the treatment step 320, the cfDNA molecule 312 is converted to generate a converted cfDNA molecule 322. During the treatment 320, the second CpG site which was unmethylated has its cytosine converted to uracil. However, the first and third CpG sites were not converted.

After conversion, a sequencing library 330 is prepared and sequenced 340 to generate a sequence read 342. The analytics system aligns 350 the sequence read 342 to a reference genome 344. The reference genome 344 provides the context as to what position in a human genome the fragment cfDNA originates from. In this simplified example, the analytics system aligns 350 the sequence read 342 such that the three CpG sites correlate to CpG sites 33, 24, and 25 (arbitrary reference identifiers used for convenience of description). The analytics system can thus generate information both on methylation status of all CpG sites on the cfDNA molecule 312 and the position in the human genome that the CpG sites map to. As shown, the CpG sites on sequence read 342 which are methylated are read as cytosines. In this example, the cytosines appear in the sequence read 342 only in the first and third CpG site which allows one to infer that the first and third CpG sites in the original cfDNA molecule are methylated. Whereas, the second CpG site can be read as a thymine (U is converted to T during the sequencing process), and thus, one can infer that the second CpG site is unmethylated in the original cfDNA molecule. With these two pieces of information, the methylation status and location, the analytics system generates 360 a methylation state vector 352 for the fragment cfDNA 312. In this example, the resulting methylation state vector 352 is <M₂₃, U₂₄, M₂₅>, wherein M corresponds to a methylated CpG site, U corresponds to an unmethylated CpG site, and the subscript number corresponds to a position of each CpG site in the reference genome.

One or more alternative sequencing methods can be used for obtaining sequence reads from nucleic acids in a biological sample. The one or more sequencing methods can comprise any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and Nanopore sequencing can also be used to obtain sequence reads from the nucleic acids (e.g., cell-free nucleic acids) in the biological sample. Sequencing-by-synthesis and reversible terminator-based sequencing (e.g., Illumina's Genome Analyzer; Genome Analyzer II; HISEQ 3000; HISEQ 4500 (Illumina, San Diego Calif.)) can be used to obtain sequence reads from the cell-free nucleic acid obtained from a biological sample of a training subject in order to form the genotypic dataset. Millions of cell-free nucleic acid (e.g., DNA) fragments can be sequenced in parallel. In one example of this type of sequencing technology, a flow cell is used that contains an optically transparent slide with eight individual lanes on the surfaces of which are bound oligonucleotide anchors (e.g., adaptor primers). A cell-free nucleic acid sample can include a signal or tag that facilitates detection. The acquisition of sequence reads from the cell-free nucleic acid obtained from the biological sample can include obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

The one or more sequencing methods can comprise a whole-genome sequencing assay. A whole-genome sequencing assay can comprise a physical assay that generates sequence reads for a whole genome or a substantial portion of the whole genome which can be used to determine large variations such as copy number variations or copy number aberrations. Such a physical assay may employ whole-genome sequencing techniques or whole-exome sequencing techniques. A whole-genome sequencing assay can have an average sequencing depth of at least 1×, 2×, 3×, 4×, 5×, 6×, 7×, 8×, 9×, 10×, at least 20×, at least 30×, or at least 40× across the genome of the test subject. In some embodiments, the sequencing depth is about 30,000×. The one or more sequencing methods can comprise a targeted panel sequencing assay. A targeted panel sequencing assay can have an average sequencing depth of at least 50,000×, at least 55,000×, at least 60,000×, or at least 70,000× sequencing depth for the targeted panel of genes. The targeted panel of genes can comprise between 450 and 500 genes. The targeted panel of genes can comprise a range of 500±5 genes, a range of 500±10 genes, or a range of 500±25 genes.

The one or more sequencing methods can comprise paired-end sequencing. The one or more sequencing methods can generate a plurality of sequence reads. The plurality of sequence reads can have an average length ranging between 10 and 700, between 50 and 400, or between 100 and 300. The one or more sequencing methods can comprise a methylation sequencing assay. The methylation sequencing can be i) whole-genome methylation sequencing or ii) targeted DNA methylation sequencing using a plurality of nucleic acid probes. For example, the methylation sequencing is whole-genome bisulfite sequencing (e.g., WGBS). The methylation sequencing can be a targeted DNA methylation sequencing using a plurality of nucleic acid probes targeting the most informative regions of the methylome, a unique methylation database and prior prototype whole-genome and targeted sequencing assays.

The methylation sequencing can detect one or more 5-methylcytosine (5mC) and/or 5-hydroxymethylcytosine (5hmC) in respective nucleic acid methylation fragments. The methylation sequencing can comprise conversion of one or more unmethylated cytosines or one or more methylated cytosines, in respective nucleic acid methylation fragments, to a corresponding one or more uracils. The one or more uracils can be detected during the methylation sequencing as one or more corresponding thymines. The conversion of one or more unmethylated cytosines or one or more methylated cytosines can comprise a chemical conversion, an enzymatic conversion, or combinations thereof.

For example, bisulfite conversion involves converting cytosine to uracil while leaving methylated cytosines (e.g., 5-methylcytosine or 5-mC) intact. In some DNA, about 95% of cytosines may not be methylated in the DNA, and the resulting DNA fragments may include many uracils which are represented by thymines. Enzymatic conversion processes may be used to treat the nucleic acids prior to sequencing, which can be performed in various ways. One example of a bisulfite-free conversion comprises a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines. The methylation state of a CpG site in the corresponding plurality of CpG sites in the respective nucleic acid methylation fragment can be methylated when the CpG site is determined by the methylation sequencing to be methylated, and unmethylated when the CpG site is determined by the methylation sequencing to not be methylated.

A methylation sequencing assay (e.g., WGBS and/or targeted methylation sequencing) can have an average sequencing depth including but not limited to up to about 1,000×, 2,000×, 3,000×, 5,000×, 10,000×, 15,000×, 20,000×, or 30,000×. The methylation sequencing can have a sequencing depth that is greater than 30,000×, e.g., at least 40,000× or 50,000×. A whole-genome bisulfite sequencing method can have an average sequencing depth of between 20× and 50×, and a targeted methylation sequencing method has an average effective depth of between 100× and 1000×, where effective depth can be the equivalent whole-genome bisulfite sequencing coverage for obtaining the same number of sequence reads obtained by targeted methylation sequencing.

For further details regarding methylation sequencing (e.g., WGBS and/or targeted methylation sequencing), see, e.g., U.S. patent application Ser. No. 16/352,602, entitled “Methylation Fragment Anomaly Detection,” filed Mar. 13, 2019, U.S. patent application Ser. No. 16/719,902, entitled “Systems and Methods for Estimating Cell Source Fractions Using Methylation Information,” filed Dec. 18, 2019, and U.S. patent application Ser. No. 17/191,914, titled “Systems and Methods for Cancer Condition Determination Using Autoencoders,” filed Mar. 4, 2021. Other methods for methylation sequencing, including those disclosed herein and/or any modifications, substitutions, or combinations thereof, can be used to obtain fragment methylation patterns. A methylation sequencing can be used to identify one or more methylation state vectors, as described, for example, in U.S. patent application Ser. No. 16/352,602, entitled “Anomalous Fragment Detection and Classification,” filed Mar. 13, 2019, or according to any of the techniques disclosed in U.S. patent application Ser. No. 15/931,022, entitled “Model-Based Featurization and Classification,” filed May 13, 2020. Each reference is incorporated by reference in its entirety.

The methylation sequencing of nucleic acids and the resulting one or more methylation state vectors can be used to obtain a plurality of nucleic acid methylation fragments. Each corresponding plurality of nucleic acid methylation fragments (e.g., for each respective genotypic dataset) can comprise more than 100 nucleic acid methylation fragments. An average number of nucleic acid methylation fragments across each corresponding plurality of nucleic acid methylation fragments can comprise 1000 or more nucleic acid methylation fragments, 5000 or more nucleic acid methylation fragments, 10,000 or more nucleic acid methylation fragments, 20,000 or more nucleic acid methylation fragments, or 30,000 or more nucleic acid methylation fragments. An average number of nucleic acid methylation fragments across each corresponding plurality of nucleic acid methylation fragments can be between 10,000 nucleic acid methylation fragments and 50,000 nucleic acid methylation fragments. The corresponding plurality of nucleic acid methylation fragments can comprise one thousand or more, ten thousand or more, 100 thousand or more, one million or more, ten million or more, 100 million or more, 500 million or more, one billion or more, two billion or more, three billion or more, four billion or more, five billion or more, six billion or more, seven billion or more, eight billion or more, nine billion or more, or 10 billion or more nucleic acid methylation fragments. An average length of a corresponding plurality of nucleic acid methylation fragments can be between 140 and 480 nucleotides.

### III. Deconvolution of Component Proportions

Deconvolution of component proportions involves determining a breakdown of component proportions where nucleic acid fragments in a sample originate from. The component proportions represent a breakdown of components contributing nucleic acid fragments to the sample. In one or more embodiments, the component proportions specify a percentage of the sample attributed to each component. For example, the deconvolution process determines a first percentage of nucleic acid fragments in a sample originating from a first component, a second percentage of nucleic acid fragments in the sample originating from a second component, so on and so forth with remaining components. In other embodiments, the component proportions may specify a rank of the components according to the predicted proportions. For example, component 3 is the largest contributor of nucleic acid fragments to the sample, followed by component 1, then component 2 (in an example with the mixture model predicting proportions for three components).

The deconvolution process models methylation signatures of various components. A methylation signature represents the methylation sequence reads of the component. In some embodiments, the methylation signature comprises counts of methylation variants over a plurality of genomic regions. In other embodiments, the methylation signature may further comprise counts of reference states over the plurality of genomic regions. The reference state at a genomic region may include remaining methylation patterns not assigned to variant states. As for the components, each component may be a tissue type, a cell type, or further subtypes thereof.

The deconvolution process may be applied at various stages of the overall cancer classification workflow 100 described in FIG. 1. In one example implementation, the deconvolution process may be utilized to assess sample purity, e.g., at step 142 of the workflow 100. In another example implementation, the deconvolution process may be utilized in the cancer classification 146 to determine a particular cancer type. As yet another example implementation, the deconvolution process may be utilized to quantify cancer signal, e.g., for use in monitoring cancer status and/or progression. Other example implementations may utilize the component proportions in other manners.

### III.A. Mixture Model

The analytics system may train a mixture model for deconvolving component proportions. The mixture model generally predicts component proportions based on the methylation sequence reads of a sample. The mixture model generally comprises a plurality of component submodels. A component submodel models the methylation signature of a component. The number of components to deconvolve in a set of training samples may be tuned as a hyperparameter of the mixture model. Upon training, the component submodel may input a methylation signature of the sample and output a component likelihood indicating a likelihood that the methylation signature of the sample is derived from the component.

FIGS. 4A & 4B illustrate an overview of utilizing a mixture model. The analytics system (or its various components) may perform the methods of FIGS. 4A & 4B. In other embodiments, other general computing devices may perform any of the steps of the methods. In other embodiments, the methods may include additional steps, fewer steps, different steps, or some combination thereof.

FIG. 4A is an exemplary flowchart describing a method 400 of training a mixture model to predict component proportions in a sample, according to one or more embodiments.

The analytics system obtains 410 a plurality of training samples. Each training sample may comprise methylation sequence reads, e.g., as generated by sample sequencing 120 in FIG. 1, or more specifically sample processing 300 of FIG. 3A. Each methylation sequence read may include methylation information of a nucleic acid fragment of a sample. A sample may be a liquid biopsy sample, a tissue-derived sample, an isolated cell sample, another biological sample, or some combination thereof. The samples may include circulating tumor cells, disseminated tumor cells, other types of somatic cells, etc. The training samples may have known or unknown component proportions. For example, a first sample may be a tissue-derived sample that includes nucleic acid fragments originating from the isolated tissue. The first sample would have a component proportion that is predominantly the isolated tissue. Another sample may be a purified plasma sample from a healthy subject comprising predominantly cell free nucleic acid fragments. This sample may have a predominant component proportion for non-cancer cell free nucleic acid fragments. In some example implementations, the analytics system obtains 1,000 training samples, 2,000 training samples, 3,000 training samples, 4,000 training samples, 5,000 training samples, 6,000 training samples, 7,000 training samples, 8,000 training samples, 9,000 training samples, 10,000 training samples, 20,000 training samples, 30,000 training samples, 40,000 training samples, 50,000 training samples, or more than 50,000 training samples.

The analytics system generates 420 a methylation signature for each training sample, e.g., by counting methylation variants over a plurality of genomic regions. The analytics system may segment the genome into the various genomic regions. Each genomic region may span a plurality of methylation sites, e.g., CpG sites. The analytics system may span the genome, i.e., using whole genome sequencing of the entire human genome for human training samples. In other embodiments, the methylation sequence reads are generated from a targeted methylation assay that targets a subset of genomic regions in the genome. The analytics system may also filter out genomic regions having an average sequencing depth across training samples below a threshold depth. The methylation signature may be based on other types of methylation features (e.g., methylation density at each of a plurality of methylation sites, methylation density over a genomic region, count of highly methylated fragments overlapping a genomic region, count of highly unmethylated fragments overlapping a genomic region, etc.)

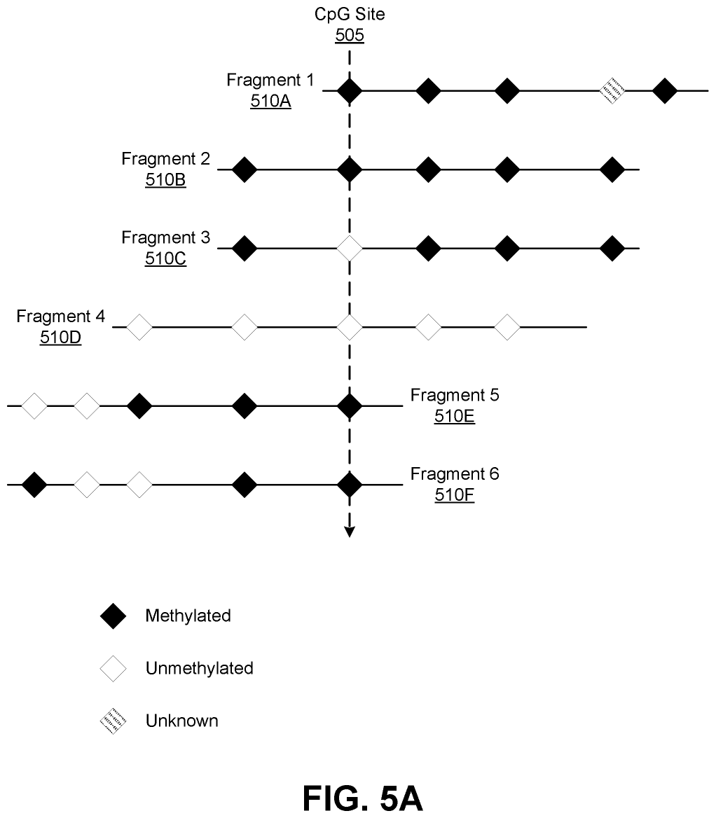

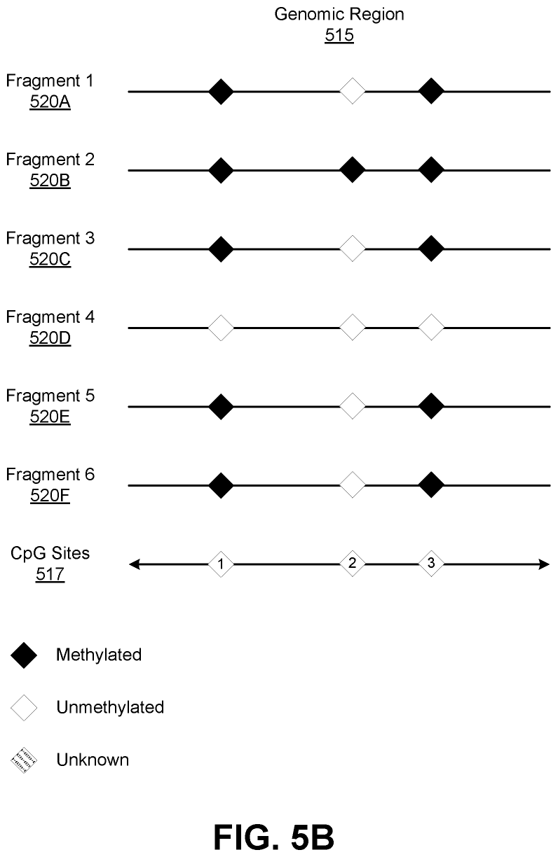

At each genomic region, the analytics system may count methylation sequence reads, each of which may be characterized as being one of a plurality of methylation variants. For example, a set of CpGs may be associated with multiple methylation variants, each of which is indicative of different cancers. Each methylation variant can have a defined variant state. In instances with two variant states, one variant state may be deemed the primary variant state, whereas the other variant state may be deemed the secondary variant state. The analytics system may count a first count of methylation sequence reads (or nucleic acid fragments) having the primary variant state and a second count of methylation sequence reads (or nucleic acid fragments) having the secondary variant state. In instances with more than two variant states, there may be the primary variant state, a secondary variant state, and multiple other variant states. The analytics system may count methylation sequence reads having each of the variant states, i.e., a first count for the primary variant state, and a count for each other variant state. In other embodiments, the analytics system may count a first count for the primary variant state and a second count for the one or more other variant states together. In some embodiments, the analytics system may count just the alternate variant states. FIGS. 5A & 5B illustrate counting of variant states.

In one or more embodiments, the analytics system may perform genomic region selection. The analytics system may exclude genomic regions according to one or more selection criteria. One example selection criterium sets a limit on the number of methylation variants for a genomic region to be included. For example, if more than two methylation variants occur at a genomic region in significant proportions within a population, then the analytics system may exclude such genomic region. The significant proportion, which also may be referred to as prevalence, may be 1%, 2%, 3%, 4%, 5%, 6%, 7%, 8%, 9%, 10%, 11%, 12%, 13%, 14%, 15%, 20%, 25%, or 30%. Another selection criterium may rank and exclude genomic regions according to their discriminatory power between the components. For example, the analytics system, when training the mixture model, may generate an information gain score for each genomic region, based on the genomic region's informativeness in the deconvolution. Genomic regions having below a threshold score may be excluded.

FIG. 5A illustrates counting methylation variants from a single CpG site 505 as a genomic region, according to one or more embodiments. In a single CpG site genomic region, there generally are two methylation states, a reference state and a variant state (pertaining to the methylation variant). The analytics system may count a first count of methylation sequence reads having the reference state and a second count of methylation sequence reads having the variant state.

For example, in FIG. 5A, there are six fragments 510 that overlap the single CpG site 505. At each CpG site on a fragment, denoted as a diamond, the fragment has a methylation state. Methylation states may include methylated, shown as filled in, unmethylated shown as unfilled, and unknown shown with a diagonal hatch. Unknown methylation may include indetermined states caused from mutations or sequencing errors. As a single site, there are two possible methylation states at this genomic region (M or U). One methylation state is deemed the reference state while the other is deemed the variant state. For example, at the CpG site 505 as the genomic region, the reference genome provides that this CpG site predominantly is methylated, thus the reference state is methylated. As for counts, the analytics system may count four fragments (or methylation sequence reads) as having the reference state of methylated, and two fragments (or methylation sequence reads) as having the variant state of unmethylated.

FIG. 5B illustrates counting methylation variants from a genomic region 515 spanning multiple CpG sites 517, according to one or more embodiments. The CpG sites 517 include CpG sites 1, 2, and 3. As in FIG. 5A, a filled diamond indicates methylation, an unfilled diamond indicates unmethylation, and a diagonal hatch diamond indicates unknown. In the embodiment shown, fragment 1 520A may have a methylation pattern comprising methylated at CpG site 1, unmethylated at CpG site 2, and methylated at CpG site 3. As an example, this may be a reference state. The analytics system may count the number of fragments having the reference state of methylated, unmethylated, and methylated at the genomic region 515. In this example, four fragments have the reference state. Fragment 2 520B has a methylation variant, that is different than the reference state. Fragment 2 520B has a methylation pattern with all three CpG site 517 methylated. The analytics system may count one total fragment (or methylation sequence read) as having this first methylation variant. Fragment 4 520D has another methylation variant, that is different than the reference state and the first methylation variant. Fragment 4 520D has a methylation pattern with all three CpG sites 517 unmethylated. The analytics system may count one total fragment (or methylation sequence read) as having this second methylation variant. The analytics system may also sum counts of all methylation variants. In such example, the count for all methylation variants is two, including fragment 2 520A having the first methylation variant and fragment 4 520D having the second methylation variant.

Returning to FIG. 4A, the analytics system generates 420 a methylation signature based on the counts of the methylation variants. In one embodiment, the methylation signature is a count of methylation variant(s) for each of a plurality of genomic regions. In another embodiment, the methylation signature is a percentage of methylation variant(s) for each of the plurality of genomic regions. To calculate the percentage of methylation variant(s) for a genomic region, the analytics system counts a total count of methylation sequence reads present at the genomic region and a count of methylation sequence reads having the alternate methylation variant(s). The percentage equals the count of methylation sequence reads having the methylation variant(s) over the total count of methylation sequence reads present at the genomic region. For example, there are 10,000 genomic regions, and the methylation signature is a vector of length 10,000. A first value of the vector corresponds to a count or percentage for methylation variant(s) at the first genomic region, a second value of the vector corresponds to a count or percentage for methylation variant(s) at the second genomic region, and so on through the 10,000 genomic regions.

In some embodiments, the methylation signature may further include a count or percentage of the reference states for each genomic region. In an example with 10,000 genomic regions, the methylation signature would comprise 20,000 values including 10,000 counts or percentages of reference states and 10,000 counts or percentages of methylation variants across the genomic regions.

In some embodiments, the methylation signature may further include one or more counts or percentages for each methylation variant at a genomic region. For example, a population at one genomic region statistically expresses two or more alternate methylation variants in significant proportions of the population (e.g., above 5%, 10%, 15%, etc.). The methylation signature may also include a count or percentage for each methylation variant at that genomic region, e.g., a first count for a first methylation variant, a second count for a second methylation variant with different methylation pattern from the first methylation variant, and so on with other distinct methylation variants.

In one or more embodiments, the methylation signature may be normalized. Normalizing may be based on sequencing depth of the sample. Normalizing based on sequencing depth normalizes variance in genetic material across the samples.

The analytic system trains 430 the mixture model with the methylation signatures of the training samples. The analytics system may train the mixture model to predict component proportions (also referred to as deconvolution of component proportions) based on an input methylation signature. The analytics system may perform a maximum likelihood estimation to train the model to fit the methylation signatures of the training samples.

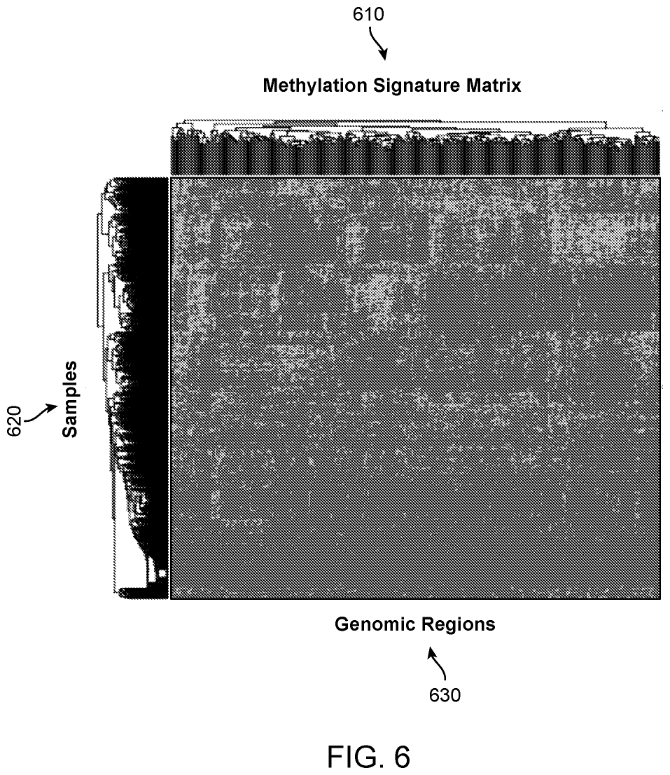

The analytics system may train the mixture model as a machine-learning model. Training of the machine-learning model may include supervised training, unsupervised training, or semi-supervised training. Supervised training generally entails utilizing a known label or value that can be used to calculate an error or loss of the model's prediction. The analytics system training the model may adjust parameters of the model to minimize the error. With the mixture model, the analytics system may utilize training samples having known component proportions to provide the supervised loss in training the mixture model. Unsupervised training generally entails learning of patterns in the training data without known labels or values supervising the learning. Example unsupervised techniques include clustering, anomaly detection, latent-variable learning, other types of machine-learning techniques that do not rely on known labels or values, etc. In the context of the mixture model, the analytics system may utilize training samples without known component proportions and allow the mixture model to learn patterns within the training data. Semi-supervised learning may generally entails utilizing training data with some of the training data having known labels or values. Types of machine-learning models that may be implemented include decision trees, neural networks, multilayer perceptrons, support vector machine, models relying on other types of machine-learning, derivatives thereof, and combinations thereof. Embodiments of the mixture model architecture are described in FIGS. 6 & 7.

FIG. 6 illustrates an example methylation signature matrix 610 used in training the mixture model, according to an example implementation. The methylation signature matrix 610 represents the methylation signatures of the training samples used for training the mixture model. The methylation signature matrix 610 comprises a plurality of samples 620 over a plurality of genomic regions 630. The methylation signature of each training sample 620 is represented in a row of the methylation signature matrix 610.

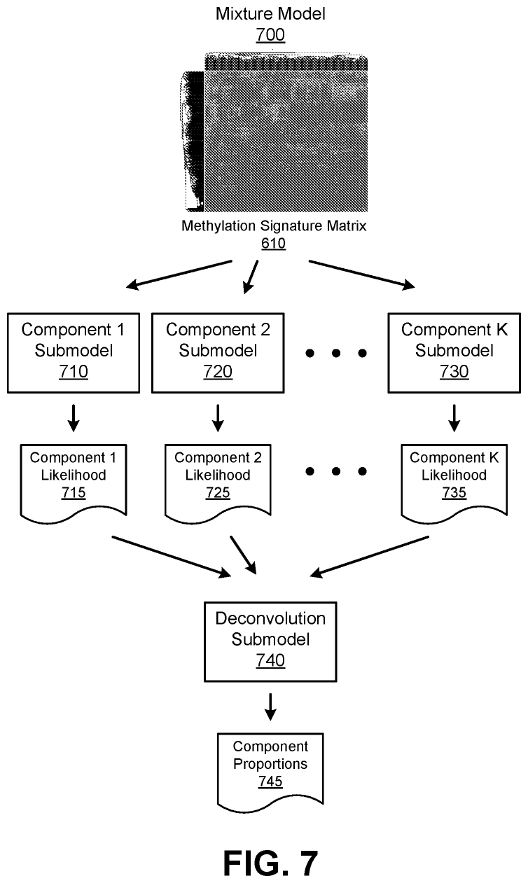

FIG. 7 illustrates an exemplary architecture of the mixture model 700, according to one or more embodiments. The mixture model 700 comprises a plurality of component submodels and a deconvolution model 740. Each component submodel is configured to input the methylation signature of a sample and to output a predicted likelihood that the methylation signature originates from the component. The predicted likelihood may be represented as a percentage, e.g., 65% likelihood that the methylation signature originates from component 1. For example, component 1 submodel 710 outputs a component 1 likelihood 715, component 2 submodel 720 outputs a component 2 likelihood 725, so on with component submodels up to component K submodel 730 outputs a component K likelihood 735. The component likelihoods are input into a deconvolution that outputs component proportions for the methylation signature.

The analytics system may train the component submodels and the deconvolution submodel 740 concurrently. Concurrent training entails inputting the methylation signature matrix 610 through all the submodels concurrently, with the analytics system adjusting parameters of the various submodels concurrently. In a supervised learning approach to training, the analytics system may feed one or more training samples through the mixture model 700 to predict component proportions for the training samples. The analytics system may calculate a loss for a training sample by a comparison of the sample's known component proportions against the sample's predicted component proportions. In an unsupervised learning approach to training, the analytics system may feed the training samples through the mixture model 700 to cluster like methylation signatures as predominantly origination from the same component. In such unsupervised approaches, the analytics system may be agnostic to the training sample's true component proportions.

In other embodiments, the analytics system may separately train one or more of the submodels. Separate training entails utilizing a subset of the training data to train a submodel. Once trained, the analytics system may fix the parameters of the submodel while training other submodels. For example, the analytics system may train each of the component submodels separately, then hold the parameters of the component submodels while training the deconvolution submodel 740. The analytics system may utilize training samples that predominantly originate from one component to train that component's corresponding component submodel. Then the analytics system may utilize training samples with mixed proportions to train the deconvolution model 740.

In one or more embodiments, the mixture model 700 may comprise further division of a component submodel into further subcomponent submodels. For example, the component 1 submodel 710 may be connected to one or more subcomponent submodels (not shown). A subcomponent submodel may further determine a subcomponent likelihood that the methylation signature is attributed to the subcomponent. For example, component 1 may represent breast tissue shed from potential breast tumor cells. The component 1 may comprise human epidermal growth factor receptor 2 (HER2) positive and negative status as further division of the breast tissue. The component 1 submodel 710 may provide a component 1 likelihood 715 of 65% breast tissue. A first subcomponent submodel may output, based on the methylation signature of the sample, that, if the methylation signature originates from breast tissue (component 1), the methylation signature has a 35% likelihood to be HER2 positive status. The deconvolution submodel 740 inputs the component likelihoods and any subcomponent likelihoods to determine component and subcomponent proportions. Following the above example, the deconvolution submodel 740 of the mixture model 700 may output a proportion for breast tissue as component 1, with a proportion for HER2 positive status. In some embodiments with a plurality of subcomponents, the deconvolution submodel 740 may provide a subcomponent proportion for each subcomponent, with the subcomponent proportions totaling the component proportion. The mixture model 700 may comprise a tree of components and subcomponents spanning multiple layers in depth. For example, three layers of depth may include a first component including a set of subcomponents and one of the subcomponents including a second set of subcomponents. The analytics system may separately train the subcomponent submodels, or may train the subcomponent submodels concurrently with other submodels of the mixture model 700.

Returning to FIG. 4A, the analytics system, in training the mixture model 430, may tune 435 the number of components K as a hyperparameter. K represents the number of components that the mixture model predicts component proportions for (e.g., as shown in FIG. 7). The analytics system may iteratively train the mixture model while adjusting the number of components K while cross-validating the trained mixture model. Cross-validation may include utilizing a validation set of training samples with known component proportions. The analytics system may input the methylation signatures of the training samples to predict the component proportions. The analytics system may determine an error or a loss as a difference between the known component proportions and the predicted component proportions. The analytics system may score each trained mixture model with different K number of components based on the errors or the losses from the validation set. In one or more embodiments, the analytics system may apply a penalization as a factor of the number of components, so as to induce the mixture model to optimize the number of components. Too little components and the model may poorly fit the training data. Too many components and the model may overfit the training data. The analytics system may also score how well the mixture model fits to the training data.

In one or more embodiments, the analytics system may inform the mixture model what each component corresponds to. In such embodiments, the analytics system may train the mixture model without knowing labels of the training data. The analytics system may later utilize known labels or proportions of the training data to inform what each component corresponds to. The analytics system may utilize a training sample with known component proportions, for example, a training sample that contains 65% of nucleic acid fragments originating from breast tissue and 35% of nucleic acid fragments corresponding to non-cancer cell-free nucleic acid fragments. The analytics system may input the training sample into the mixture model, which outputs the predicted components proportions of component 1 to be 70%, component 2 to be 25%, and component 3 to be 5% (in an example mixture model trained over at least three components). The analytics system may match the largest known component proportion and the largest predicted component proportion to be corresponding to the same component. The analytics system may do likewise with the other known component proportions and the other predicted component proportions in the training sample. The analytics system may utilize a plurality of labeled training samples to corroborate the labeling of the components in the mixture model. For example, the majority of training samples suggest matching component 1 of the mixture model to lung tissue, and so on for the remaining components of the mixture model.

FIG. 4B is an exemplary flowchart describing a method 440 of deploying a mixture model to predict component proportions in a sample, according to one or more embodiments. The mixture model may be trained according to the method 400 of training the mixture model.

The analytics system obtains 450 a test sample for predicting component proportions. The test sample comprises genetic material including methylation information. The analytics system may or may not know a disease or cancer status of the subject providing the test sample. The methylation information may include methylation sequence reads, e.g., as sequenced by the process described in FIGS. 3A & 3B.

The analytics system generates 460 a methylation signature for the test sample by counting methylation variants over the plurality of genomic regions. The plurality of genomic regions matches to the plurality of genomic regions the mixture model is trained on. The analytics system, akin to the training samples, may normalize the test sample's methylation signature, e.g., based on sequencing depth.

The analytics system applies 470 the mixture model to the methylation signature to predict component proportions for the test sample. The analytics system inputs the methylation signature of the test sample into the mixture model which outputs the component proportions.

### III.B. Applications of the Mixture Model

Based on how the mixture model is trained, the analytics system may employ the mixture model in a variety of applications.

In one or more embodiments, the mixture model may be utilized for tissue purity assessment (e.g., in step 142 of FIG. 1). The analytics system may utilize the mixture model to evaluate sample purity prior to use of that sample, e.g., for training a cancer classifier, etc. For example, the analytics system may obtain a plurality of training samples diagnosed with specific cancer types. The analytics system may utilize the mixture model to predict component proportions of the training samples. In one embodiment, the analytics system may exclude training samples having below a threshold proportion of tissue relating to that specific cancer type. For example, a training sample originates from a subject diagnosed with lung cancer, but the mixture model predicts the training sample to have 10% of nucleic acid fragments originating from lung tissue. If the analytics system is retaining training samples having above 30% of nucleic acid fragments originating from the corresponding tissue of the diagnosed cancer type, then the analytics system may withhold use of that training sample having below the threshold when training the cancer classifier. This can advantageously focus training of a cancer classifier on training samples with substantial cancer signal, thereby improving the sensitivity of the cancer classifier. Utilizing the mixture model for tissue purity can also identify training samples with misdiagnoses. For example, a training sample originating from a subject diagnosed with prostate cancer may have a large contribution of liver tissue in the liquid biopsy sample, as determined by the mixture model. The analytics system may withhold such training samples in the training of the cancer classifier, to prevent skewing the cancer classifier.

In one or more embodiments, the mixture model may be utilized to predict a cancer type of a test subject. The analytics system may utilize the mixture model subsequent to or as part of a cancer classifier. The analytics system may utilize the cancer classifier to generate a cancer prediction. The cancer prediction may include a binary status (between cancer and non-cancer), a cancer type (between a plurality of cancer types), or some combination thereof. In one embodiment, in response to predicting a likelihood of presence of cancer (a binary status), the analytics system may utilize the mixture model to determine component proportions as a manner of determining a cancer type. The largest component proportion that is not non-cancer or other healthy tissue may be determined to be the test sample's cancer type. In other embodiments, the analytics system may utilize the mixture model's component proportions to corroborate the cancer classifier's predicted cancer type. For example, the cancer classifier predicts a test sample to likely have head/neck cancer. The analytics system may utilize the mixture model to identify whether head/neck tissue is present in a substantial proportion in the test sample. If not, then the analytics system may make note of the discrepancy, e.g., lowering confidence in the prediction. The analytics system may also utilize the mixture model to determine whether there may be presence of multiple cancer types. For example, if the mixture model is providing substantial proportions of two different components (that are not non-cancer or other healthy tissue), then the analytics system may determine that the test sample may likely have two cancer types.

In one or more embodiments, the mixture model may be utilized to quantify cancer signal in a test subject. The analytics system may utilize the mixture model to predict component proportions for a test subject diagnosed with or predicted to likely have cancer. The analytics system may quantify the cancer signal based on the component proportions. In some embodiments, the analytics system may utilize the component likelihoods output by the component submodels to quantify the cancer signal. Quantification of the cancer signal may provide insight as to what stage the cancer may be at or how aggressive a cancer is. For example, the analytics system may, at different points in time, analyze test samples from an individual to assess change in cancer signal, e.g., based on the component proportions predicted by the mixture model. If the analytics system identifies increase in the cancer signal, then the analytics system may determine the cancer to be progressing. The analytics system may further determine an aggressiveness of the cancer based on the rate of increase in cancer signal. The analytics system may also use quantification of cancer signal to assess various treatment plans. If there is slowed growth or decreased quantification, then the analytics system may determine that the treatment plan being implemented is successful. Otherwise, the analytics system may determine that the treatment plan is unsuccessful, and may further recommend adjusting the treatment plan. A recommendation to adjust the treatment plan may include changing therapies, changing dosage or regimen, increasing length or frequency of treatment plan, etc.

In one or more embodiments, the analytics system may utilize the component submodels apart from the mixture model. In general, a component submodel represents a distribution of methylation variant allele fractions of genetic material from a component. As such, the analytics system may utilize a component submodel to predict likelihood that a methylation signature originates from the component. The analytics system may also generate the distribution of the methylation signatures for the component. With the component submodels, the analytics system can predict from what component a methylation sequence read originated. The analytics system may utilize such predictions to filter cancer training samples to remove non-cancer impurity sequence reads to retain the methylation sequence reads more likely to pertain to cancer signal when training of the cancer classifier. The non-cancer impurity may comprise one or more of: lymphocytes, macrophages, fibroblasts, vascular endothelial cells, or non-cancer tissue. In one or more embodiments, the analytics system may retrieve the methylation signature of the non-cancer impurity from a reference database. The reference database may include a distribution of observed methylation signatures from non-cancer tissue or cells, e.g., from healthy subjects not diagnosed with cancer (or any other genetically based disease). In some embodiments, the reference database may be populated by methylation signatures observed during prior assessments of genetic material. In some embodiments, the reference database may be populated with values retrieved from a third party, such as from a public collection of methylation signatures.

In one or more embodiments, the analytics system may extract learned information from the mixture model. In one embodiment, the analytics system may learn correlations between methylation variants at genomic regions contributing to component proportions. For example, the analytics system may identify methylation variants that are informative of particular components. In another embodiment, the analytics system may identify methylation variants at genomic regions correlated with non-cancer impurity. The analytics system may filter out use of such methylation variants at those genomic regions as those genomic regions may be confounded with non-cancer impurity.

### IV. Cancer Classifier for Determining Cancer

Cancer classification involves extraction genetic features and applying one or more models to the extracted features to determine a cancer prediction. The analytics system aggregates extracted features into a feature vector which can then be input into a trained cancer classifier to determine a cancer prediction based on the input feature vector. The cancer prediction may comprise a label and/or a value. The label may be binary, indicating a presence or absence of cancer in the test subject, and/or multiclass, indicating one or more particular cancer types from a plurality of screened cancer types. The value may be a gradation of cancer status, e.g., a stage of cancer, or quantification of cancer signal. In particular, a cancer classifier may be a machine-learned model comprising a plurality of classification parameters and a function representing a relation between the feature vector as input and the cancer prediction as output. Inputting the feature vector into the function with the classification parameters yields the cancer prediction. In one or more embodiments, cancer classification utilizes a plurality of models. Some models may be used to determine one or more features from the methylation sequence reads for use in cancer classification. Prior to deployment of the cancer classifier, the analytics system trains the cancer classifier.

### IV.A. Identifying Anomalous Fragments

The analytics system can determine anomalous fragments for a sample using the sample's methylation state vectors. For each fragment in a sample, the analytics system can determine whether the fragment is an anomalous fragment using the methylation state vector corresponding to the fragment. In some embodiments, the analytics system calculates a p-value score for each methylation state vector describing a probability of observing that methylation state vector or other methylation state vectors even less probable in the healthy control group. The analytics system may determine fragments with a methylation state vector having below a threshold p-value score as anomalous fragments. In some embodiments, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

### IV.A.I. P-Value Filtering

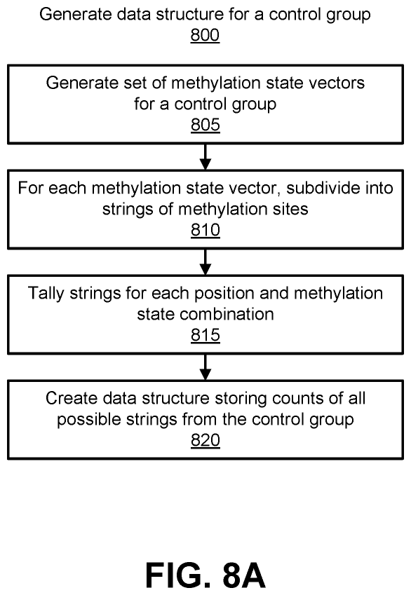

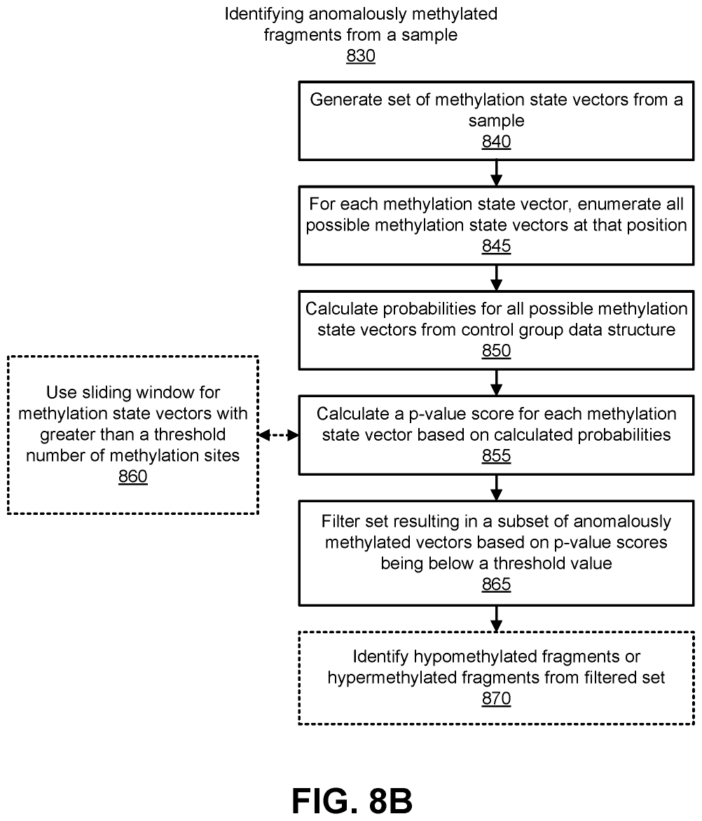

In some embodiments, the analytics system calculates a p-value score for each methylation state vector compared to methylation state vectors from fragments in a healthy control group. The p-value score can describe a probability of observing the methylation status matching that methylation state vector or other methylation state vectors even less probable in the healthy control group. In order to determine a DNA fragment to be anomalously methylated, the analytics system can use a healthy control group with a majority of fragments that are normally methylated. When conducting this probabilistic analysis for determining anomalous fragments, the determination can hold weight in comparison with the group of control subjects that make up the healthy control group. To ensure robustness in the healthy control group, the analytics system may select some threshold number of healthy individuals to source samples including DNA fragments. FIG. 8A below describes the method of generating a data structure for a healthy control group with which the analytics system may calculate p-value scores. FIG. 8B describes the method of calculating a p-value score with the generated data structure.

FIG. 8A is a flowchart describing a process 800 of generating a data structure for a healthy control group, according to an embodiment. To create a healthy control group data structure, the analytics system can receive a plurality of DNA fragments (e.g., cfDNA) from a plurality of healthy individuals. The analytics system can generate 805 a methylation state vector for each fragment, for example via the process 300.

With each fragment's methylation state vector, the analytics system can subdivide 810 the methylation state vector into strings of CpG sites. In some embodiments, the analytics system subdivides 810 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 can result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 815 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 810 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 815 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size can help keep the data structure creation and performance (e.g., use for later accessing as described below), in terms of computational and storage, reasonable. Second, a statistical consideration to limiting the maximum string length can be to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it uses a significant amount of data that may not be available, and thus can be too sparse for a model to perform appropriately. For example, calculating a probability of anomalousness/cancer conditioned on the prior 100 CpG sites can use counts of strings in the data structure of length 100, ideally some matching exactly the prior 100 methylation states. If only sparse counts of strings of length 100 are available, there can be insufficient data to determine whether a given string of length of 100 in a test sample is anomalous or not.

FIG. 8B is a flowchart describing a process 830 for identifying anomalously methylated fragments from an individual, according to an embodiment. In process 830, the analytics system generates 840 methylation state vectors from cfDNA fragments of the subject, e.g., via the process 300. The analytics system can handle each methylation state vector as follows.

For a given methylation state vector, the analytics system enumerates 845 all possibilities of methylation state vectors having the same starting CpG site and same length (i.e., set of CpG sites) in the methylation state vector. As each methylation state is generally either methylated or unmethylated there can be effectively two possible states at each CpG site, and thus the count of distinct possibilities of methylation state vectors can depend on a power of 2, such that a methylation state vector of length n would be associated with 2ⁿ possibilities of methylation state vectors. With methylation state vectors inclusive of indeterminate states for one or more CpG sites, the analytics system may enumerate 830 possibilities of methylation state vectors considering only CpG sites that have observed states.

The analytics system calculates 850 the probability of observing each possibility of methylation state vector for the identified starting CpG site and methylation state vector length by accessing the healthy control group data structure. In some embodiments, calculating the probability of observing a given possibility uses a Markov chain probability to model the joint probability calculation. The Markov model can be trained, at least in part, based upon evaluation of a methylation state of each CpG site in the corresponding plurality of CpG sites of the respective fragment (e.g., nucleic acid methylation fragment) across those nucleic acid methylation fragments in a healthy noncancer cohort dataset that have the corresponding plurality of CpG sites. For example, a Markov model (e.g., a Hidden Markov Model or HMM) is used to determine the probability that a sequence of methylation states (comprising, e.g., “M” or “U”) can be observed for a nucleic acid methylation fragment in a plurality of nucleic acid methylation fragments, given a set of probabilities that determine, for each state in the sequence, the likelihood of observing the next state in the sequence. The set of probabilities can be obtained by training the HMM. Such training can involve computing statistical parameters (e.g., the probability that a first state can transition to a second state (the transition probability) and/or the probability that a given methylation state can be observed for a respective CpG site (the emission probability)), given an initial training dataset of observed methylation state sequences (e.g., methylation patterns). HMMs can be trained using supervised training (e.g., using samples where the underlying sequence as well as the observed states are known) and/or unsupervised training (e.g., Viterbi learning, maximum likelihood estimation, expectation-maximization training, and/or Baum-Welch training). In other embodiments, calculation methods other than Markov chain probabilities are used to determine the probability of observing each possibility of methylation state vector. For example, such calculation method can include a learned representation. The p-value threshold can be between 0.01 and 0.10, or between 0.03 and 0.06. The p-value threshold can be 0.05. The p-value threshold can be less than 0.01, less than 0.001, or less than 0.0001.

The analytics system calculates 855 a p-value score for the methylation state vector using the calculated probabilities for each possibility. In some embodiments, this includes identifying the calculated probability corresponding to the possibility that matches the methylation state vector in question. Specifically, this can be the possibility having the same set of CpG sites, or similarly the same starting CpG site and length as the methylation state vector. The analytics system can sum the calculated probabilities of any possibilities having probabilities less than or equal to the identified probability to generate the p-value score.

This p-value can represent the probability of observing the methylation state vector of the fragment or other methylation state vectors even less probable in the healthy control group. A low p-value score can, thereby, generally correspond to a methylation state vector which is rare in a healthy individual, and which causes the fragment to be labeled anomalously methylated, relative to the healthy control group. A high p-value score can generally relate to a methylation state vector is expected to be present, in a relative sense, in a healthy individual. If the healthy control group is a non-cancerous group, for example, a low p-value can indicate that the fragment is anomalous methylated relative to the non-cancer group, and therefore possibly indicative of the presence of cancer in the test subject.

As above, the analytics system can calculate p-value scores for each of a plurality of methylation state vectors, each representing a cfDNA fragment in the test sample. To identify which of the fragments are anomalously methylated, the analytics system may filter 865 the set of methylation state vectors based on their p-value scores. In some embodiments, filtering is performed by comparing the p-values scores against a threshold and keeping only those fragments below the threshold. This threshold p-value score can be on the order of 0.1, 0.01, 0.001, 0.0001, or similar.

According to example results from the process 800, the analytics system can yield a median (range) of 2,800 (1,500-12,000) fragments with anomalous methylation patterns for participants without cancer in training, and a median (range) of 3,000 (1,200-420,000) fragments with anomalous methylation patterns for participants with cancer in training. These filtered sets of fragments with anomalous methylation patterns may be used for the downstream analyses as described below in Section IV.B.

In some embodiments, the analytics system uses 860 a sliding window to determine possibilities of methylation state vectors and calculate p-values. Rather than enumerating possibilities and calculating p-values for entire methylation state vectors, the analytics system can enumerate possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window can identify the sequential set of CpG sites from the vector within the window starting from the first CpG site in the vector. The analytic system can calculate a p-value score for the window including the first CpG site. The analytics system can then “slide” the window to the second CpG site in the vector, and calculates another p-value score for the second window. Thus, for a window size l and methylation vector length m, each methylation state vector can generate m−l+1 p-value scores. After completing the p-value calculations for each portion of the vector, the lowest p-value score from all sliding windows can be taken as the overall p-value score for the methylation state vector. In other embodiments, the analytics system aggregates the p-value scores for the methylation state vectors to generate an overall p-value score.

Using the sliding window can help to reduce the number of enumerated possibilities of methylation state vectors and their corresponding probability calculations that would otherwise need to be performed. To give a realistic example, it can be for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations can enumerate 2\hat{ }5 (32) possibilities of methylation state vectors, which total results in 50×2\hat{ }5 (1.6×10\hat{ }3) probability calculations. This can result in a vast reduction of calculations to be performed, with no meaningful hit to the accurate identification of anomalous fragments.

In embodiments with indeterminate states, the analytics system may calculate a p-value score summing out CpG sites with indeterminates states in a fragment's methylation state vector. The analytics system can identify all possibilities that have consensus with the all methylation states of the methylation state vector excluding the indeterminate states. The analytics system may assign the probability to the methylation state vector as a sum of the probabilities of the identified possibilities. As an example, the analytics system can calculate a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states can use calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In some embodiments, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities can allow for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

One or more nucleic acid methylation fragments can be filtered prior to training region models or cancer classifier. Filtering nucleic acid methylation fragments can comprise removing, from the corresponding plurality of nucleic acid methylation fragments, each respective nucleic acid methylation fragment that fails to satisfy one or more selection criteria (e.g., below or above one selection criteria). The one or more selection criteria can comprise a p-value threshold. The output p-value of the respective nucleic acid methylation fragment can be determined, at least in part, based upon a comparison of the corresponding methylation pattern of the respective nucleic acid methylation fragment to a corresponding distribution of methylation patterns of those nucleic acid methylation fragments in a healthy noncancer cohort dataset that have the corresponding plurality of CpG sites of the respective nucleic acid methylation fragment.

Filtering a plurality of nucleic acid methylation fragments can comprise removing each respective nucleic acid methylation fragment that fails to satisfy a p-value threshold. The filter can be applied to the methylation pattern of each respective nucleic acid methylation fragment using the methylation patterns observed across the first plurality of nucleic acid methylation fragments. Each respective methylation pattern of each respective nucleic acid methylation fragment (e.g., Fragment One, . . . , Fragment N) can comprise a corresponding one or more methylation sites (e.g., CpG sites) identified with a methylation site identifier and a corresponding methylation pattern, represented as a sequence of 1's and 0's, where each “1” represents a methylated CpG site in the one or more CpG sites and each “0” represents an unmethylated CpG site in the one or more CpG sites. The methylation patterns observed across the first plurality of nucleic acid methylation fragments can be used to build a methylation state distribution for the CpG site states collectively represented by the first plurality of nucleic acid methylation fragments (e.g., CpG site A, CpG site B, . . . , CpG site ZZZ). Further details regarding processing of nucleic acid methylation fragments are disclosed in U.S. Provisional patent application Ser. No. 17/191,914, titled “Systems and Methods for Cancer Condition Determination Using Autoencoders,” filed Mar. 4, 2021, which is hereby incorporated herein by reference in its entirety.

The respective nucleic acid methylation fragment may fail to satisfy a selection criterion in the one or more selection criteria when the respective nucleic acid methylation fragment has an anomalous methylation score that is less than an anomalous methylation score threshold. In this situation, the anomalous methylation score can be determined by a mixture model. For example, a mixture model can detect an anomalous methylation pattern in a nucleic acid methylation fragment by determining the likelihood of a methylation state vector (e.g., a methylation pattern) for the respective nucleic acid methylation fragment based on the number of possible methylation state vectors of the same length and at the same corresponding genomic location. This can be executed by generating a plurality of possible methylation states for vectors of a specified length at each genomic location in a reference genome. Using the plurality of possible methylation states, the number of total possible methylation states and subsequently the probability of each predicted methylation state at the genomic location can be determined. The likelihood of a sample nucleic acid methylation fragment corresponding to a genomic location within the reference genome can then be determined by matching the sample nucleic acid methylation fragment to a predicted (e.g., possible) methylation state and retrieving the calculated probability of the predicted methylation state. An anomalous methylation score can then be calculated based on the probability of the sample nucleic acid methylation fragment.

The respective nucleic acid methylation fragment can fail to satisfy a selection criterion in the one or more selection criteria when the respective nucleic acid methylation fragment has less than a threshold number of residues. The threshold number of residues can be between 10 and 50, between 50 and 100, between 100 and 150, or more than 150. The threshold number of residues can be a fixed value between 20 and 90. The respective nucleic acid methylation fragment may fail to satisfy a selection criterion in the one or more selection criteria when the respective nucleic acid methylation fragment has less than a threshold number of CpG sites. The threshold number of CpG sites can be 4, 5, 6, 7, 8, 9, or 10. The respective nucleic acid methylation fragment can fail to satisfy a selection criterion in the one or more selection criteria when a genomic start position and a genomic end position of the respective nucleic acid methylation fragment indicates that the respective nucleic acid methylation fragment represents less than a threshold number of nucleotides in a human genome reference sequence.

The filtering can remove a nucleic acid methylation fragment in the corresponding plurality of nucleic acid methylation fragments that has the same corresponding methylation pattern and the same corresponding genomic start position and genomic end position as another nucleic acid methylation fragment in the corresponding plurality of nucleic acid methylation fragments. This filtering step can remove redundant fragments that are exact duplicates, including, in some instances, PCR duplicates. The filtering can remove a nucleic acid methylation fragment that has the same corresponding genomic start position and genomic end position and less than a threshold number of different methylation states as another nucleic acid methylation fragment in the corresponding plurality of nucleic acid methylation fragments. The threshold number of different methylation states used for retention of a nucleic acid methylation fragment can be 1, 2, 3, 4, 5, or more than 5. For example, a first nucleic acid methylation fragment having the same corresponding genomic start and end position as a second nucleic acid methylation fragment but having at least 1, at least 2, at least 3, at least 4, or at least 5 different methylation states at a respective CpG site (e.g., aligned to a reference genome) is retained. As another example, a first nucleic acid methylation fragment having the same methylation state vector (e.g., methylation pattern) but different corresponding genomic start and end positions as a second nucleic acid methylation fragment is also retained.

The filtering can remove assay artifacts in the plurality of nucleic acid methylation fragments. The removal of assay artifacts can comprise removing sequence reads obtained from sequenced hybridization probes and/or sequence reads obtained from sequences that failed to undergo conversion during bisulfite conversion. The filtering can remove contaminants (e.g., due to sequencing, nucleic acid isolation, and/or sample preparation).

The filtering can remove a subset of methylation fragments from the plurality of methylation fragments based on mutual information filtering of the respective methylation fragments against the cancer state across the plurality of training subjects. For example, mutual information can provide a measure of the mutual dependence between two conditions of interest sampled simultaneously. Mutual information can be determined by selecting an independent set of CpG sites (e.g., within all or a portion of a nucleic acid methylation fragment) from one or more datasets and comparing the probability of the methylation states for the set of CpG sites between two sample groups (e.g., subsets and/or groups of genotypic datasets, biological samples, and/or subjects). A mutual information score can denote the probability of the methylation pattern for a first condition versus a second condition at the respective region in the respective frame of the sliding window, thus indicating the discriminative power of the respective region. A mutual information score can be similarly calculated for each region in each frame of the sliding window as it progresses across the selected sets of CpG sites and/or the selected genomic regions. Further details regarding mutual information filtering are disclosed in U.S. patent application Ser. No. 17/119,606, titled “Cancer Classification using Patch Convolutional Neural Networks,” filed Dec. 11, 2020, which is hereby incorporated herein by reference in its entirety.

### IV.A.II. Hypermethylated Fragments and Hypomethylated Fragments

In some embodiments, the analytics system identifies 870 determines hypomethylated fragments or hypermethylated fragments from the filtered set as anomalous fragments. The analytics system identifies hypermethylated fragments having over a threshold number of CpG sites and over a threshold percentage of the CpG sites methylated. The analytics system identifies hypomethylated fragments having over the threshold number of CpG sites and over a threshold percentage of CpG sites unmethylated. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### IV.B. Training of Cancer Classifier

FIG. 9A is a flowchart describing a process 900 of training a cancer classifier, according to an embodiment. The analytics system obtains 910 a plurality of training samples each having a set of anomalous fragments and a label of a cancer type. The plurality of training samples can include any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 920, for each training sample, a feature vector based on the set of anomalous fragments of the training sample. The analytics system can calculate an anomaly score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragment in a sample that overlap the CpG site and calculates an anomaly score based on the count of 5. In one or more embodiments, the feature vector further includes one or more features based on the methylation variants, e.g., as identified and counted in FIGS. 4A & 4B for use in the mixture model.

Once all anomaly scores are determined for a training sample, the analytics system can determine the feature vector as a vector of elements including, for each element, one of the anomaly scores associated with one of the CpG sites in an initial set. The analytics system can normalize the anomaly scores of the feature vector based on a coverage of the sample. Here, coverage can refer to a median or average sequencing depth over all CpG sites covered by the initial set of CpG sites used in the classifier, or based on the set of anomalous fragments for a given training sample.

As an example, reference is now made to FIG. 9B illustrating a matrix of training feature vectors 922. In this example, the analytics system has identified CpG sites [S] 926 for consideration in generating feature vectors for the cancer classifier. The analytics system selects training samples [N] 924. The analytics system determines a first anomaly score 928 for a first arbitrary CpG site [s1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 928 for the first CpG site as 1, as illustrated in FIG. 9B. Considering a second arbitrary CpG site [s2], the analytics system similarly checks the set of anomalous fragments for at least one that includes the second CpG site [s2]. If the analytics system does not find any such anomalous fragment that includes the second CpG site, the analytics system determines a second anomaly score 929 for the second CpG site [s2] to be 0, as illustrated in FIG. 9B. Once the analytics system determines all the anomaly scores for the initial set of CpG sites, the analytics system determines the feature vector for the first training sample [n1] including the anomaly scores with the feature vector including the first anomaly score 928 of 1 for the first CpG site [s1] and the second anomaly score 929 of 0 for the second CpG site [s2] and subsequent anomaly scores, thus forming a feature vector [1, 0, . . . ].

Additional approaches to featurization of a sample can be found in: U.S. application Ser. No. 15/931,022 entitled “Model-Based Featurization and Classification;” U.S. application Ser. No. 16/579,805 entitled “Mixture Model for Targeted Sequencing;” U.S. application Ser. No. 16/352,602 entitled “Anomalous Fragment Detection and Classification;” and U.S. application Ser. No. 16/723,716 entitled “Source of Origin Deconvolution Based on Methylation Fragments in Cell-Free DNA Samples;” all of which are incorporated by reference in their entirety.

The analytics system may further limit the CpG sites considered for use in the cancer classifier. The analytics system computes 930, for each CpG site in the initial set of CpG sites, an information gain based on the feature vectors of the training samples. From step 920, each training sample has a feature vector that may contain an anomaly score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 930 an information gain for each cancer type and for each CpG site in the initial set to determine whether to include that CpG site in the classifier. The information gain is computed for training samples with a given cancer type compared to all other samples. For example, two random variables ‘anomalous fragment’ (‘AF’) and ‘cancer type’ (‘CT’) are used. In one embodiment, AF is a binary variable indicating whether there is an anomalous fragment overlapping a given CpG site in a given samples as determined for the anomaly score/feature vector above. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site. In practice, for a first cancer type, the analytics system computes pairwise mutual information gain against each other cancer type and sums the mutual information gain across all the other cancer types.

For a given cancer type, the analytics system can use this information to rank CpG sites based on how cancer specific they are. This procedure can be repeated for all cancer types under consideration. If a particular region is commonly anomalously methylated in training samples of a given cancer but not in training samples of other cancer types or in healthy training samples, then CpG sites overlapped by those anomalous fragments can have high information gains for the given cancer type. The ranked CpG sites for each cancer type can be greedily added (selected) 940 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 950 the feature vectors of the training samples as needed. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 920 or to the selected set of CpG sites from step 950. In one embodiment, the analytics system trains 960 a binary cancer classifier to distinguish between cancer and non-cancer based on the feature vectors of the training samples. In this manner, the analytics system uses training samples that include both non-cancer samples from healthy individuals and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 970 a multiclass cancer classifier to distinguish between many cancer types (also referred to as tissue of origin (TOO) labels). Cancer types can include one or more cancers and may include a non-cancer type (may also include any additional other diseases or genetic disorders, etc.). To do so, the analytics system can use the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood. The analytics system may further corroborate the TOO prediction by utilizing the mixture model of FIGS. 4A & 4B to predict component proportions of a sample. Further description above in FIG. 4B.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

The classifier can include a logistic regression algorithm, a neural network algorithm, a support vector machine algorithm, a Naive B ayes algorithm, a nearest neighbor algorithm, a boosted trees algorithm, a random forest algorithm, a decision tree algorithm, a multinomial logistic regression algorithm, a linear model, or a linear regression algorithm.

### IV.C. Deployment of Cancer Classifier

During use of the cancer classifier, the analytics system can obtain a test sample from a subject of unknown cancer type. The analytics system may process the test sample comprised of DNA molecules with any combination of the processes 300 and 830 to achieve a set of anomalous fragments. The analytics system can determine a test feature vector for use by the cancer classifier according to similar principles discussed in the process 900. The analytics system can calculate an anomaly score for each CpG site in a plurality of CpG sites in use by the cancer classifier. For example, the cancer classifier receives as input feature vectors inclusive of anomaly scores for 1,000 selected CpG sites. The analytics system can thus determine a test feature vector inclusive of anomaly scores for the 1,000 selected CpG sites based on the set of anomalous fragments. The analytics system can calculate the anomaly scores in a same manner as the training samples. In some embodiments, the analytics system defines the anomaly score as a binary score based on whether there is a hypermethylated or hypomethylated fragment in the set of anomalous fragments that encompasses the CpG site. The analytics system may generate the test feature vector including one or more features based on the methylation variants (as described in FIGS. 4A & 4B).

The analytics system can then input the test feature vector into the cancer classifier. The function of the cancer classifier can then generate a cancer prediction based on the classification parameters trained in the process 900 and the test feature vector. In the first manner, the cancer prediction can be binary and selected from a group consisting of “cancer” or non-cancer;” in the second manner, the cancer prediction is selected from a group of many cancer types and “non-cancer.” In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 960 of the process 900 with another cancer classifier trained in step 970 or the process 900. The analytics system can input the test feature vector into the cancer classifier trained as a binary classifier in step 960 of the process 900. The analytics system can receive an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier can receive the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system can determine a cancer score for a test sample based on the test sample's sequencing data (e.g., methylation sequencing data, SNP sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

The classifier may be used to determine the disease state of a test subject, e.g., a subject whose disease status is unknown. The method can include obtaining a test genomic data construct (e.g., single time point test data), in electronic form, that includes a value for each genomic characteristic in the plurality of genomic characteristics of a corresponding plurality of nucleic acid fragments in a biological sample obtained from a test subject. The method can then include applying the test genomic data construct to the test classifier to thereby determine the state of the disease condition in the test subject. The test subject may not be previously diagnosed with the disease condition.

The classifier can be a temporal classifier that uses at least (i) a first test genomic data construct generated from a first biological sample acquired from a test subject at a first point in time, and (ii) a second test genomic data construct generated from a second biological sample acquired from a test subject at a second point in time.

The trained classifier can be used to determine the disease state of a test subject, e.g., a subject whose disease status is unknown. In this case, the method can include obtaining a test time-series data set, in electronic form, for a test subject, where the test time-series data set includes, for each respective time point in a plurality of time points, a corresponding test genotypic data construct including values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points. The method can then include applying the test genotypic data construct to the test classifier to thereby determine the state of the disease condition in the test subject. The test subject may not be previously diagnosed with the disease condition.

### V. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

### V.A. Early Detection of Cancer

In some embodiments, the methods and/or classifier of the present invention are used to detect the presence or absence of cancer in a subject suspected of having cancer. For example, a classifier (e.g., as described above in Section IV.) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### V.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed according to the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

### V.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy). The physician can prescribe an appropriate treatment based on analyses performed by the analytics system, e.g., the analyses 140 of FIG. 1.

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### VI. Kit Implementation

Also disclosed herein are kits for performing the methods described above including the methods relating to the cancer classifier. The kits may include one or more collection vessels for collecting a sample from the individual comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions, particular mutations, particular genetic variants, or some combination thereof. In other embodiments, samples collected via the kit are provided to a sequencing laboratory that may use the sequencing panels to sequence the nucleic acids in the sample.

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illumine how to operate a computing device as the analytics system 200, for the purposes of performing the steps of any of the methods or processes described.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address which can be used via the internet to access the information at a removed site.

### VII. Example Results

### VII.A. Sample Collection and Processing

Study design and samples: CCGA (NCT02889978) is a prospective, multi-center, case-control, observational study with longitudinal follow-up. De-identified biospecimens were collected from approximately 15,000 participants from 342 sites. Samples were divided into training (1,785) and test (1,015) sets; samples were selected to ensure a prespecified distribution of cancer types and non-cancers across sites in each cohort, and cancer and non-cancer samples were frequency age-matched by gender.

Whole-genome bisulfite sequencing: cfDNA was isolated from plasma, and whole-genome bisulfite sequencing (WGBS; 30× depth) was employed for analysis of cfDNA. cfDNA was extracted from two tubes of plasma (up to a combined volume of 10 ml) per patient using a modified QlAamp Circulating Nucleic Acid kit (Qiagen; Germantown, MD). Up to 75 ng of plasma cfDNA was subjected to bisulfite conversion using the EZ-96 DNA Methylation Kit (Zymo Research, D5003). Converted cfDNA was used to prepare dual indexed sequencing libraries using Accel-NGS Methyl-Seq DNA library preparation kits (Swift BioSciences; Ann Arbor, MI) and constructed libraries were quantified using KAPA Library Quantification Kit for Illumina Platforms (Kapa Biosystems; Wilmington, MA). Four libraries along with 10% PhiX v3 library (Illumina, FC-110-3001) were pooled and clustered on an Illumina NovaSeq 7000 S2 flow cell followed by 150-bp paired-end sequencing (30×).

For each sample, the WGBS fragment set was reduced to a small subset of fragments having an anomalous methylation pattern. Additionally, hyper or hypomethylated cfDNA fragments were selected. cfDNA fragments selected for having an anomalous methylation pattern and being hyper or hypermethylated, i.e., UFXM. Fragments occurring at high frequency in individuals without cancer, or that have unstable methylation, are unlikely to produce highly discriminatory features for classification of cancer status. We therefore produced a statistical model and a data structure of typical fragments using an independent reference set of 108 non-smoking participants without cancer (age: 58±14 years, 79 [73%] women) (i.e., a reference genome) from the CCGA study. These samples were used to train a Markov-chain model (order 3) estimating the likelihood of a given sequence of CpG methylation statuses within a fragment. This model was demonstrated to be calibrated within the normal fragment range (p-value>0.001) and was used to reject fragments with a p-value from the Markov model as >=0.001 as insufficiently unusual.

As described above, further data reduction step selected only fragments with at least 5 CpGs covered, and average methylation either >0.9 (hyper methylated) or <0.1 (hypo-methylated). This procedure resulted in a median (range) of 2,800 (1,500-12,000) UFXM fragments for participants without cancer in training, and a median (range) of 3,000 (1,200-420,000) UFXM fragments for participants with cancer in training. As this data reduction procedure only used reference set data, this stage was only required to be applied to each sample once.

### VII.B. Mixture Model Results

The following mixture model results arose from mixture models trained and deployed according to the methods described in FIGS. 4A & 4B, and throughout this disclosure. The mixture models are trained using training samples comprising methylation sequence reads. The analytics system may tune or fix the number of components in the mixture model as a hyperparameter. Once trained, the analytics system utilizes a validation set of samples to validate the accuracy of the mixture model.

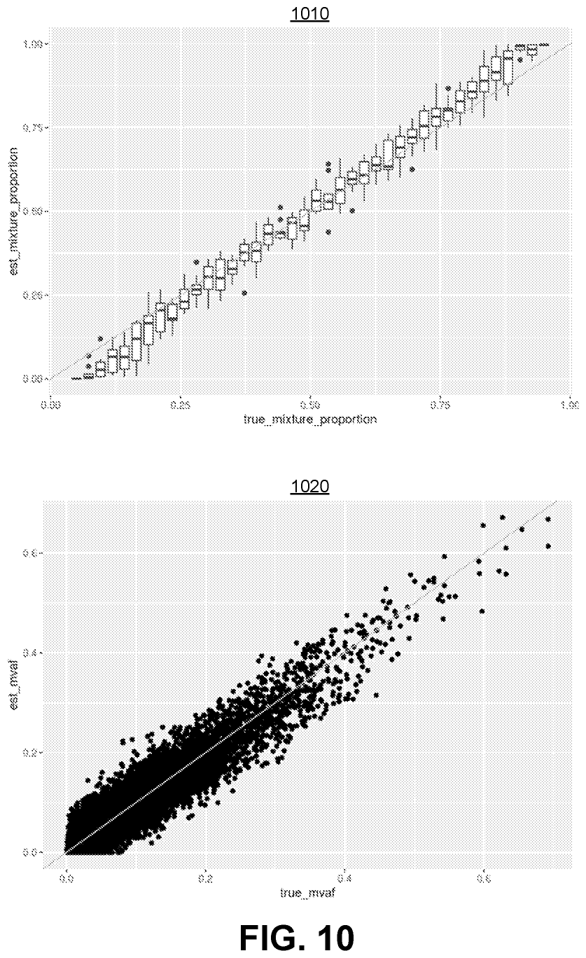

FIG. 10 is an example result of a two-component mixture model, according to an example implementation. The mixture model is trained to discern component proportions between two components. The analytics system utilizes a validation set of forty samples of varying known component proportions. The average sequence depth of the samples is 10. The mixture model evaluates over 5,000 methylation variants in the genome. The analytics system applies the trained two-component mixture model on the methylation signatures of the validation set of forty samples.

The top graph 1010 represents the accuracy of the mixture model to predict the component proportions. In the top graph 1010, the x-axis represents true component proportions between the two components for the forty validation samples, with the y-axis representing the predicted component proportions by the mixture model. The diagonal from the (0.00, 0.00) to (1.00, 1.00) represents accurate predictions of the component proportions. Qualitatively, all forty validation samples are on or touching the diagonal.

The bottom graph 1020 represents the ability of the mixture model to predict the methylation signature based on known component proportions. In graph 1020, the x-axis represents true methylation variant allele fraction, with the y-axis representing the predicted methylation variant allele fraction by the mixture model. The diagonal from the (0.00, 0.00) to (1.00, 1.00) represents accurate predictions of the methylation variant allele fraction. For a given value on the graph 1020, the accuracy of a prediction can be determined based on the deviation from the diagonal. Qualitatively, few predictions of methylation variant allele fraction deviate far from the true methylation variant allele fraction.

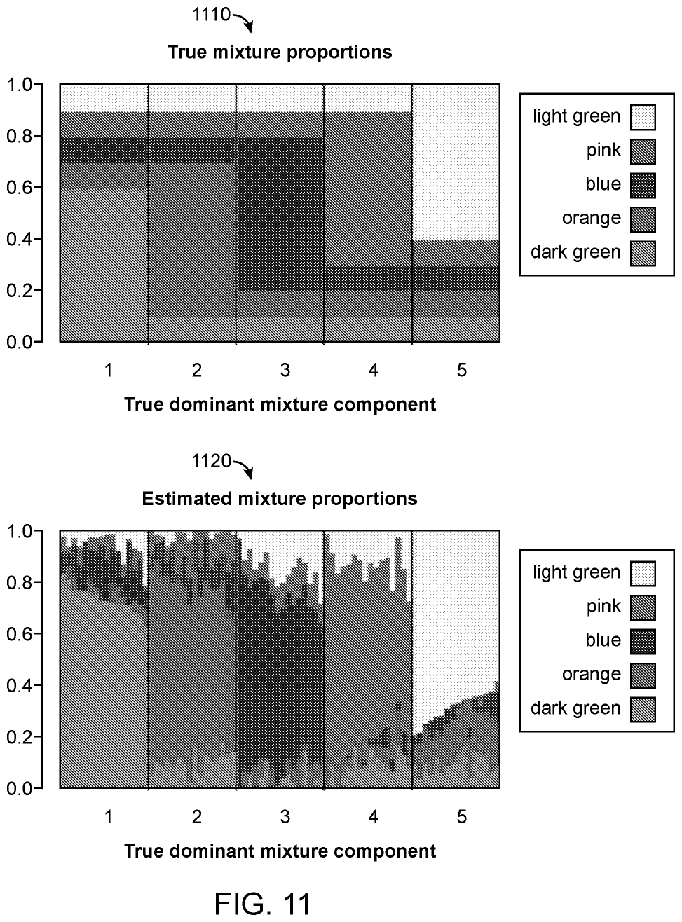

FIG. 11 is an example result of a five-component mixture model, according to an example implementation. The mixture model is trained to discern component proportions between five components. The analytics system utilizes a validation set comprising five classes of sixteen samples. Each class of samples have the same known component proportions. In particular, class 1 comprises 60% of a first component (dark green) and 10% of each of the remaining components; class 2 comprises 60% of a second component (orange) and 10% of each of the remaining components; class 3 comprises 60% of a third component (blue) and 10% of each of the remaining components; class 4 comprises 60% of a fourth component (pink) and 10% of each of the remaining components; and class 5 comprises 60% of a fifth component (light green) and 10% of each of the remaining components. The mixture model evaluates over 5,000 methylation variants in the genome. The analytics system applies the trained five-component mixture model on the methylation signatures of the validation set of the five classes of samples. The top graph 1110 illustrates the five classes of samples, with their known breakdown of component proportions. Each class is grouped together along the x-axis. The y-axis represents percentages or proportions. The bottom graph 1120 illustrates the mixture model's prediction of component proportions of the five classes. The mixture model accurately predicts that all of class 1's samples have more than 50% of the first component (dark green), that all of class 2's samples have more than 50% of the second component (orange), that all of class 3's samples have more than 50% of the third component (blue), that all of class 4's samples have more than 50% of the fourth component (pink), and that all of class 5's samples have more than 50% of the fifth component (light green). Although the validation samples may be artificially generated to have the exact component proportions, the mixture model was able to accurately identify the dominant component proportion in each of the eighty samples.

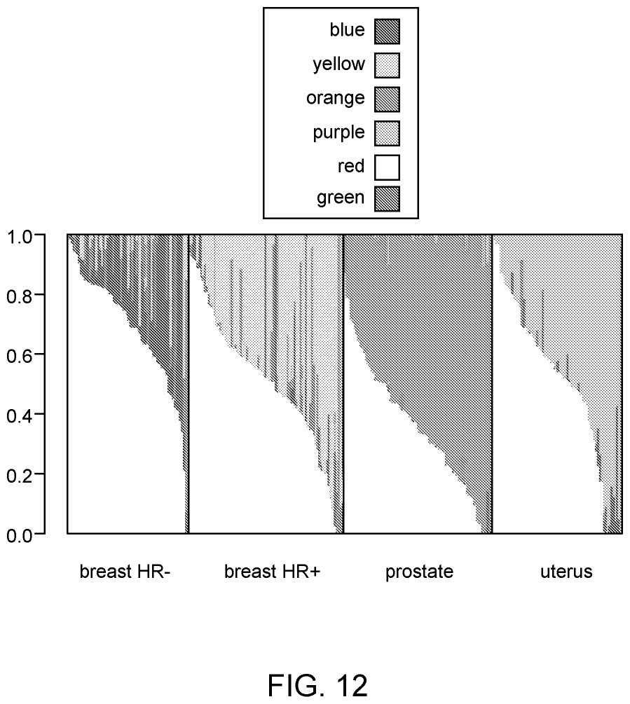

FIG. 12 is an example result of a mixture model deconvolving breast tissue positive hormone receptor status, breast tissue negative hormone receptor status, prostate tissue, and uterine tissue, according to an example implementation. The mixture model was trained allowing for the number of components to be self-defined. The mixture model is applied to validation samples known to have one of the cancer types corresponding to the components. Each class is grouped together along the x-axis. The y-axis represents percentages or proportions. The red component represents non-cancer or generic signal common across many samples. In the prostate class (all samples diagnosed with prostate cancer) and the uterus class (all samples diagnosed with uterine cancer), the mixture model predominantly predicts the samples (apart from the red component) to have a dominant proportion of the orange component (for the prostate class) and of the purple component (for the uterus class). Although the mixture model is agnostic to labels of the components, the mixture model successfully deconvolved and attributed like methylation signatures to the same component. There are a couple of uterus samples that may have predicted less of the purple component than another non-red component, but the vast majority of the uterus samples were predicted by the mixture model to have a significant contribution from the purple component. With the breast HER2− and the breast HER2+, the mixture model determined there were sufficient distinctions to warrant having two separate components between two classes. The mixture model had some challenge with some samples predicted to have a significant contribution of the other component. This could have been due to mislabeling by the healthcare provider that diagnosed the samples, or can also be due to the two tissues having similar methylation signatures.

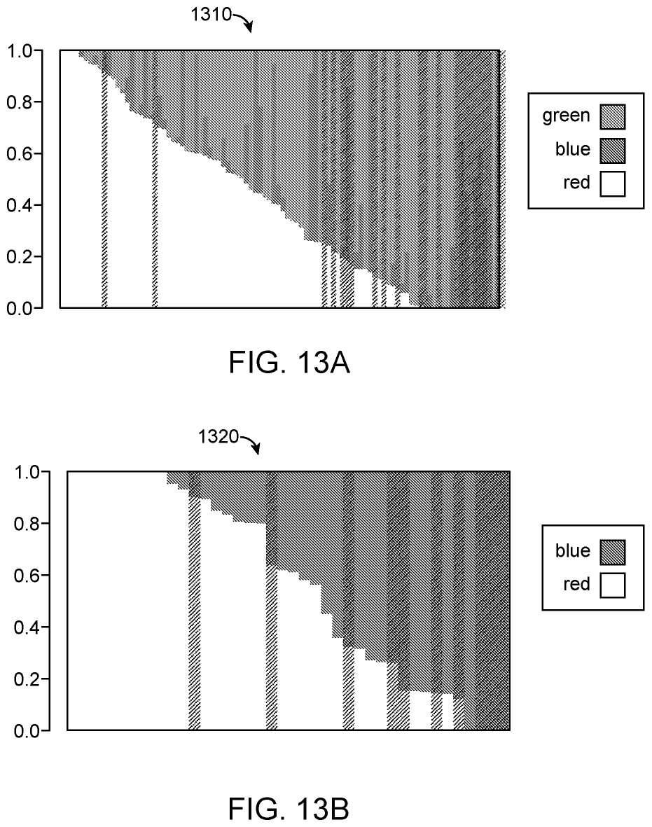

FIG. 13A is an example result of a mixture model deconvolving non-cancer impurity from colorectal tissue in colorectal samples, according to an example implementation. The analytics system, when training the mixture model, tunes the number of components to be three for the colorectal samples illustrated in graph 1310. The three components are represented by the colors red, green, and blue. The red component seems to represent a putative non-cancer impurity present in most samples. Each column represents a colorectal sample. The gray columns represent disseminating tumor cell enriched samples. Notably, the mixture model is able to assess purity of such samples. There was an improvement of purity assessment from 50% to 87% utilizing the mixture model to assess the tissue purity. The two components of blue and green likely indicate heterogeneity in the colorectal cancer tissue.

FIG. 13B is an example result of a mixture model deconvolving non-cancer impurity from bladder tissue in bladder samples, according to an example implementation. The analytics system, when training the mixture model, tunes the number of components to be two for the bladder samples illustrated in graph 1320. The two components are represented by the colors red and blue. The red component seems to represent a putative non-cancer impurity present in most samples. Each column represents a bladder sample. The gray columns represent disseminating tumor cell enriched samples. Notably, the mixture model is able to assess purity of such samples. There was an improvement of purity assessment from 19% to 67% utilizing the mixture model to assess the tissue purity.

### VIII. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method for training a machine-learned mixture model for identifying tissue types comprising:
obtaining a set of training samples comprising at least one thousand methylation sequence reads derived from sequencing deoxyribonucleic acid (DNA) fragments;
modifying each training sample to produce a corresponding sample methylation signature by:
determining, for each genomic region of a plurality of genomic regions, a first set of methylation sequence reads that overlap the genomic region,
determining a second set of methylation sequence reads that include a methylation variant at the genomic region, and
generating the sample methylation signature generated based at least in part on the first sets of methylation sequence reads and the second sets of methylation sequence reads;
generating a training set of data comprising the sample methylation signatures; and
training the machine-learned mixture model using the training set of data, the machine-learned mixture model configured to identify a contribution of each of a plurality of originating tissue types for DNA fragments in a sample.

2. The method of claim 1, wherein at least one training sample is known to comprise a first originating tissue type of the plurality of originating tissue types, wherein training the mixture model comprises training the mixture model to identify contribution of the first originating tissue type for DNA fragments in the one training sample.

3. The method of claim 1, wherein at least one training sample is known to have contribution of DNA fragments from each of the plurality of originating tissue types, wherein training the mixture model comprises training the mixture model to identify contribution of each of the plurality of originating tissue types for DNA fragments in the one training sample.

4. The method of claim 1, wherein at least one training sample is one of: a liquid biopsy sample, a tissue biopsy sample, and a purified sample.

5. The method of claim 1, wherein at least one genomic region consists of one CpG site, and at least one other genomic region comprises a plurality of CpG sites.

6. (canceled)

7. The method of claim 1, further comprising:
determining an average sequencing depth for each of an initial set of genomic regions based on the methylation sequence reads of the training samples; and
filtering out genomic regions with average sequencing depth below a threshold depth to select the plurality of genomic regions.

8. The method of claim 1, wherein the methylation variant at a genomic region is one of two or more methylation patterns at the genomic region.

9.-10. (canceled)

11. The method of claim 1, wherein modifying each training sample to produce the corresponding sample methylation signature further comprises:
determining, for each genomic region of the plurality of genomic regions, a third set of methylation sequence reads having a reference state at the genomic region, wherein the reference state is any methylation pattern not belonging to the methylation variant, wherein the sample methylation signature is generated further based on the third set of methylation sequence reads.

12. The method of claim 1, wherein the tissue types include a combination of: a non-cancer impurity; squamous cell cancer tissue; skin carcinoma tissue; melanoma tissue; lung cancer tissue; adenocarcinoma of the lung tissue; squamous carcinoma of the lung tissue; cancer of the peritoneum tissue; gastrointestinal cancer tissue; pancreatic cancer tissue; cervical cancer tissue; ovarian cancer tissue; liver cancer tissue; hepatoma tissue; hepatic carcinoma tissue; bladder cancer tissue; testicular cancer tissue; breast cancer tissue; brain cancer tissue; colon cancer tissue; rectal cancer tissue; colorectal cancer tissue; endometrial or uterine carcinoma tissue; salivary gland carcinoma tissue; kidney or renal cancer tissue; prostate cancer tissue; vulvar cancer tissue; thyroid cancer tissue; anal carcinoma tissue; penile carcinoma tissue; head and neck cancer tissue; esophageal carcinoma tissue; and nasopharyngeal carcinoma (NPC) tissue.

13. The method of claim 12, wherein the non-cancer impurity comprises one or more of lymphocytes, macrophages, fibroblasts, vascular endothelial cells, or non-cancer tissue.

14. The method of claim 12, wherein the methylation signature for the non-cancer impurity is retrieved from a reference database comprising a plurality of methylation signatures for non-cancer impurity.

15. The method of claim 1, wherein training the machine-learned mixture model is according to a maximum likelihood estimation.

16. The method of claim 1, wherein training the machine-learned mixture model comprises tuning a number of tissue types as one hyperparameter of the machine-learned mixture model.

17. The method of claim 16, wherein tuning the number of tissue types as one hyperparameter of the machine-learned mixture model comprises:
for each number of originating tissue types in a number range:
training the machine-learned mixture model having the number as the hyperparameter,
determining a maximum likelihood by cross-validating the trained machine-learned mixture model with a holdout set of samples, and
implementing a penalization to the maximum likelihood based on the number; and
selecting an optimal number from the range as the hyperparameter based on penalized maximum likelihoods.

18. (canceled)

19. The method of claim 1, wherein the machine-learned mixture model comprises a first set of tissue type models, each tissue type model modeling methylation signature of DNA fragments of an originating tissue type, and wherein training the machine-learned mixture model comprises training the first set of tissue type models.

20. The method of claim 19, wherein training the first set of tissue type models comprises training each tissue type model according to a Beta distribution.

21. The method of claim 1, wherein the machine-learned mixture model comprises a deconvolution model for deconvolving the contributions of the originating tissue types for each training sample, wherein training the machine-learned mixture model comprises training the deconvolution model.

22. The method of claim 21, wherein training the deconvolution model comprises training the deconvolution model according to a binomial distribution.

23. The method of claim 1, wherein the machine-learned mixture model comprises a first tier of one or more submodels to predict contributions of macro originating tissue types and a second tier of one or more submodels to predict contributions of originating tissue types under the macro tissue types, and wherein one first tier submodel predicts a contribution of one macro tissue type and a set of one or more second tier submodels predicts contributions of a set of tissue types under the one macro tissue type equaling the contribution of the one macro tissue type.

24.-34. (canceled)

35. A method for training a cancer classifier comprising:
obtaining a cancer cohort of training samples and a non-cancer cohort of training samples, wherein each training sample from the cancer cohort and the non-cancer cohort comprises at least one thousand methylation sequence reads for DNA fragments in the training sample;
generating a sample methylation for each training sample by:
for each of a plurality of genomic regions:
determining a first set of methylation sequence reads overlapping the genomic region,
determining a second set of methylation sequence reads having an alternative methylation signature at the genomic region, and
wherein the sample methylation signature is based in part on the first sets of methylation sequence reads and the second sets of methylation sequence reads, and
applying a machine-learning mixture model to the sample methylation signature of each training sample in the cancer cohort to identify a subset of methylation sequence reads originating from a non-cancer impurity type;
excluding, for each training sample in the cancer cohort, the subset of methylation sequence reads originating from the non-cancer impurity type resulting in a feature set of methylation sequence reads;
generating, for each training sample in the non-cancer cohort, a feature set of methylation sequence reads; and
training the cancer classifier with the feature sets of methylation sequence reads for the training samples from the cancer cohort and the feature sets of methylation sequence reads for the training samples from the non-cancer cohort.

36.-47. (canceled)

48. The method of claim 35, wherein the machine-learning mixture model is trained by:
obtaining a set of training samples comprising at least one thousand methylation sequence reads derived from sequencing deoxyribonucleic acid (DNA) fragments;
modifying each training sample to produce a corresponding sample methylation signature by:
determining, for each genomic region of a plurality of genomic regions, a first set of methylation sequence reads that overlap the genomic region,
determining a second set of methylation sequence reads that include a methylation variant at the genomic region, and
generating the sample methylation signature generated based at least in part on the first sets of methylation sequence reads and the second sets of methylation sequence reads;
generating a training set of data comprising the sample methylation signatures; and
training the machine-learned mixture model using the training set of data, the machine-learned mixture model configured to identify a contribution of each of a plurality of originating tissue types for DNA fragments in a sample.
